(19) United States
(12) Patent Application Publication
(10) Pub. No.: US 2020/0024576 A1
(43) Pub. Date: Jan. 23, 2020

(54) CARDIAC TISSUE MODELS AND METHODS OF USE THEREOF

(71) Applicant: The Regents of the University of California, Oakland, CA (US)

(72) Inventors: Kevin E. Healy, Moraga, CA (US); Zhen Ma, Berkeley, CA (US)

(21) Appl. No.: 16/335,644

(22) PCT Filed: Oct. 4, 2017
(86) PCT No.: PCT/US17/55144

§ 371 Date: Mar. 21, 2019

Related U.S. Application Data
(60) Provisional application No. 62/404,717, filed on Oct. 5, 2016.

(51) Int. Cl. C12N 5/077; G01N 33/50; G01N 3/20

(52) U.S. Cl. CPC C12N 5/0657; G01N 33/5061; G01N 3/20; C12N 2506/45; G01N 2203/028; C12N 2513/00; C12N 2535/00; G01N 2203/0096; C12N 2510/00

(57) ABSTRACT

The present disclosure provides a 3-dimensional filamentous fiber matrix, systems comprising the matrix, and methods for using the matrix and the systems.

### CROSS-REFERENCE

This application claims the benefit of U.S. Provisional Patent Application No. 62/404,717, filed Oct. 5, 2016, which application is incorporated herein by reference in its entirety.

### INTRODUCTION

The integration of complex in vitro cardiac tissue models with human induced pluripotent stem (hiPS) cells and genome editing tools has been shown to enhance the physiological phenotype, improve cardiomyocyte (CMs) maturity, and recapitulate disease pathologies.

Contraction force, a key component of cardiac function, is continuously regulated by the surrounding environment. The contraction force of cardiomyocytes (CMs) derived from human induced pluripotent stem cells (hiPS-CMs) has been deemed as one of the essential parameters for the evaluation of normal mature cardiac function, disease phenotypes, and response to pharmacological interventions. Based on deformable substrates or micro-post arrays, traction force microscopy (TFM) has been widely used for single-cell force measurement at the nano-Newton (nN) scale. Two-dimensional (2D) arrays provide high spatial resolution of the contraction forces generated by individual or sheets of CMs, but does not provide three-dimensional (3D) architecture and cell-cell interactions native at the tissue level. 3D models may deliver physiological-relevant cell microenvironments and recapitulate the dynamics of the tissue-level biological responses.

3D engineered cardiac tissues that mimic native tissue structures have been developed using a variety of methodologies and materials, which share a common process of hiPS-CMs encapsulation into external hydrogels. To promote hiPS-CMs alignment and formation of physiologically relevant tissue structures, the 3D cardiac tissues are normally anchored between two flexible cantilevers, which also serve as a force sensor to report tissue-level contraction force at micro-Newton (μN) scale. However, this measurement is compromised by the matrix mechanics of the external hydrogel, which alters the tissue mechanical properties and cellular contractile force.

In parallel, the force sensors used to measure cardiac tissue contraction not only report the contraction forces generated by hiPS-CMs, but also naturally become the external mechanical microenvironment that regulate the cardiac tissue formation, remodeling and function. In TFM, variation of substrate stiffness alters the myofibril organization of 2D micropatterned hiPS-CMs, demonstrating substrata with optimal stiffness could improve the contractile activity of hiPS-CMs. In 3D cardiac tissue models, flexible cantilevers used to anchor cardiac tissues also represent the rigidity of an external structure to anchor tissue contraction, and consequently has been used to mimic in vitro cardiac tissue afterload. Increase of the afterload to cardiac microtissues derived from patient-specific and genome-engineered hiPS cells has facilitated better modeling of dilated cardiomyopathy (DCM) associated with titin (TTN) gene mutations. In contrast, optimal mechanical load was critical for the 3D maintenance and maturation of hiPS-CMs with highly organized sarcomeres, as well as increased adherens and gap junction formation. Collectively, these studies indicate the mechanical microenvironment incorporates key niche elements that regulates of cardiac function and disease phenotypes.

There remains a need in the art for improved cardiac tissue models that better mimic native tissue structures. The present disclosure provides such improved cardiac tissue models.

### SUMMARY

The present disclosure provides a 3-dimensional filamentous fiber matrix, systems comprising the matrix, and methods for using the matrix and the systems.

### BRIEF DESCRIPTION OF THE DRAWINGS

FIG. 1A-1E depict the theoretical force calculation based on fiber deflections.

FIG. 2A-2D depict the characterization of hiPS-CMs differentiation.

FIG. 3A-3C depict the generation of 3D cardiac microtissues on filamentous matrices.

FIG. 4A-4B depict 3D cardiac microtissues assembled on filamentous matrices.

FIG. 5A-5E depict force measurement based on fiber deflection.

FIG. 6A-6D depict the calculation of sarcomere alignment index.

FIG. 7A-7C depict tension indices for MYBPC3 deficient cardiac microtissues.

FIG. 8A-8C depict the fabrication of filamentous matrices.

FIG. 9A-9E depict cardiac microtissues remodeling on filamentous matrices

FIG. 10A-10E depict calcium flux of the cardiac microtissues.

FIG. 11A-11D depict generation of a MYBPC3 null hiPS cell line.

FIG. 12A-12G depict the contraction deficits of MYBPC3 deficient cardiac microtissues.

FIG. 13A-13D depict mechanical environment altered contractile phenotype.

### DEFINITIONS

The term “induced pluripotent stem cell” (or “iPS cell”), as used herein, refers to a stem cell induced from a somatic cell, e.g., a differentiated somatic cell, and that has a higher potency than said somatic cell. iPS cells are capable of self-renewal and differentiation into mature cells, e.g., cells of mesodermal lineage or cardiomyocytes. iPS cells may also be capable of differentiation into cardiac progenitor cells.

As used herein, the term “stem cell” refers to an undifferentiated cell that that is capable of self-renewal and differentiation into one or more mature cells, e.g., cells of a mesodermal lineage, cardiomyocytes, or progenitor cells. The stem cell is capable of self-maintenance, meaning that with each cell division, one daughter cell will also be a stem cell. Stem cells can be obtained from embryonic, fetal, post-natal, juvenile or adult tissue. The term “progenitor cell”, as used herein, refers to an undifferentiated cell derived from a stem cell, and is not itself a stem cell. Some progenitor cells can produce progeny that are capable of differentiating into more than one cell type.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is a human. In some embodiments, the individual is a murine.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a three-dimensional filamentous fiber matrix” includes a plurality of such matrices and reference to “the cardiomyocyte” includes reference to one or more cardiomyocytes and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a 3-dimensional filamentous fiber matrix, systems comprising the matrix, and methods for using the matrix and the systems.

Three-Dimensional Filamentous Fiber Matrices

The present disclosure provides 3-dimensional filamentous fiber matrices in which cells can be cultured. Cells cultured on subject 3-dimensional filamentous fiber matrices may readily form cell tissues, microtissues, organoids, or become organized into groups that are readily found in their native environment. Cell tissues, microtissues, organoids, or organized groups of cells as a result of cells cultured on subject 3-dimensional filamentous fiber matrices may be useful in modeling particular tissues and organs (e.g., cardiac tissue), both in their wild type and diseased states. Subject filamentous matrices provide physiologically relevant cell microenvironments and recapitulate the dynamics of the tissue-level biological responses.

The present disclosure provides a three-dimensional filamentous fiber matrix comprising: a) a first cardiomyocyte population comprising a mutation in a gene encoding a gene product required for normal cardiomyocyte function, wherein the mutation reduces the level or the activity of the gene product; and/or b) a second cardiomyocyte population, wherein the second cardiomyocyte population is isogenic with the first cardiomyocyte population, but does not comprise the mutation. In some cases, the gene product is a cardiac myosin binding protein C polypeptide. In some cases, the mutation is a loss-of-function mutation. In some cases, the first and the second cardiomyocyte populations are human cardiomyocytes. In some cases, the first cardiomyocyte population is genetically modified to produce a polypeptide calcium reporter. In some cases, the calcium reporter is GCaMP6f. In some cases, the matrix comprises filamentous fibers having a diameter of from 2 μm to 20 μm. In some cases, the matrix comprises filamentous fibers having a diameter of from 5 μm to 10 μm. In some cases, the matrix comprises filamentous fibers, each fiber comprising a first end and a second end, wherein the first end and the second end of the fiber are attached to a solid support. In some cases, the solid support comprises glass or a non-water-soluble polymer (e.g., a plastic). In some cases, the filamentous fibers are from 450 μm to 600 μm in length in the Y-axis. In some cases, the filamentous fibers form layers spaced from about 40 μm to about 60 μm apart in the X-axis, and wherein the layers are spaced from about 25 μm to about 35 μm in the Z-axis. In some cases, the filamentous fibers have an elastic modulus of from about 160 MPa to about 200 MPa. In some cases, the filamentous fibers have an elastic modulus of from about 170 MPa to about 190 MPa. In some cases, the cardiomyocytes are present in the matrix at a density of from 1×10⁶ cells/cc to 6×10⁶ cells/cc. In some cases, the cardiomyocytes are present in the matrix at a density of from 2×10⁶ cells/cc to 5×10⁶ cells/cc.

Filamentous Fiber Matrix Features

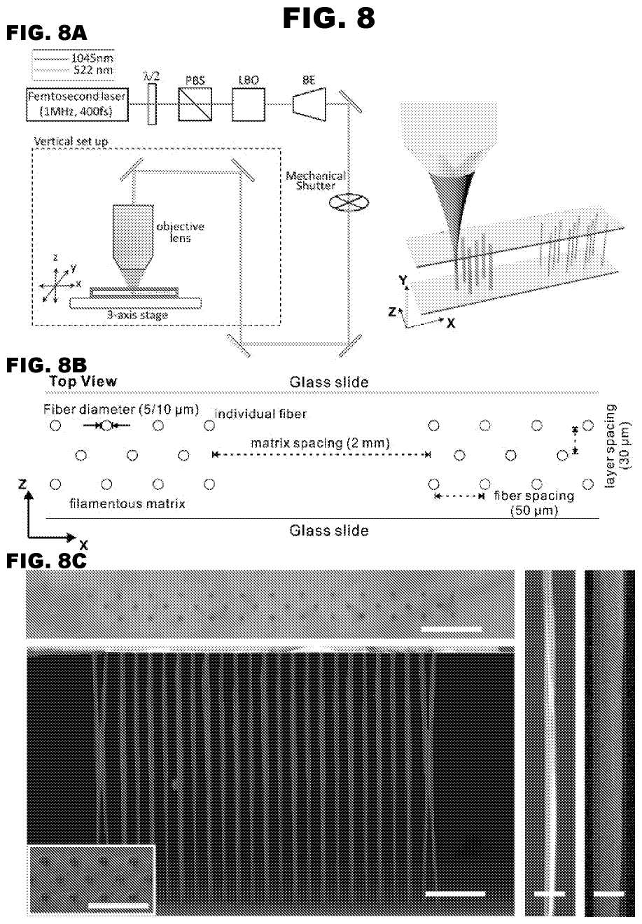

A subject 3-D filamentous fiber matrix of the present disclosure comprises a scaffold with accurately defined micro and nano-scale features. In some cases, the 3-D filamentous fiber matrix is a scaffold comprised of a plurality of fibers. In some cases, the 3-D filamentous fiber matrix is a scaffold that comprises a network of parallel fibers. In some cases, the 3-D filamentous fiber matrix is a scaffold that comprises a network of parallel and perpendicular fibers. In some cases, the 3-D filamentous fiber matrix is a scaffold that comprises a meshwork of fibers. Subject filamentous fiber matrices are three-dimensional (3D) consisting of an X-axis, Y-axis, and Z-axis as shown in FIG. 8A and FIG. 8B.

In some cases, a 3-D filamentous fiber matrix of the present disclosure is fabricated on a suitable solid support. A solid support can take any number of forms, and can be made of any of a number of materials. A solid support can be a cell culture dish, a multi-well cell culture plate, etc. A solid support can comprise glass, a water-insoluble polymer, and the like. For example, the solid support surface can comprise a material such as: polyolefins, polystyrenes, “tissue culture treated” polystyrenes, poly(alkyl)methacrylates and poly(alkyl)acrylates, poly(acrylamide), poly(ethylene glycol), poly(N-isopropyl acrylamide), polyacrylonitriles, poly(vinylacetates), poly(vinyl alcohols), chlorine-containing polymers such as poly(vinyl)chloride, polyoxymethylenes, polycarbonates, polyamides, polyimides, polyurethanes, polyvinylidene difluoride (PVDF), phenolics, amino-epoxy resins, polyesters, polyethers, polyethylene terephthalates (PET), polyglycolic acids (PGA) and other degradable polyesters, poly-(p-phenyleneterephthalamides), polyphosphazenes, polypropylenes, and silicone elastomers, as well as copolymers and combinations thereof. In some embodiments, the solid support comprises polystyrene. In some embodiments, the solid support comprises “tissue culture treated” polystyrene, e.g., polystyrene that has been treated with an oxygen plasma to generate oxygen species in the polystyrene. See, e.g., Ramsey et al. (1984) In Vitro 20:802; Beaulieu et al. (2009) Langmuir 25:7169; and Kohen et al. (2009) Biointerphases 4:69.

In some embodiments, a subject 3-D filamentous fiber matrix comprises fibers of defined length in the Y-axis. In some cases, a subject 3-D filamentous fiber matrix comprises fibers of length that can be about 50 μm, about 100 μm, about 150 μm, about 200 μm, about 250 μm, about 300 μm, about 350 μm, about 400 μm, about 450 μm, about 460 μm, about 470 μm, about 480 μm, about 490 μm, about 500 μm, about 510 μm, about 520 μm, about 530 μm, about 540 μm, about 550 μm, about 600 μm, about 650 μm, about 700 μm, about 750 μm, about 800 μm, about 850 μm, about 900 μm, about 950 μm, about 1000 μm in the Y-axis. In some cases, a subject 3-D filamentous fiber matrix comprises fibers that are 500 μm in length in the Y-axis. Any suitable fiber length may be used according to the type of cells that are desired to be grown in a subject filamentous fiber matrix. A suitable fiber length may mimic the dimensions that are found in the cell type's native environment.

In some embodiments, a subject 3-D filamentous fiber matrix comprises fibers that are spaced by a defined distance, i.e. comprises fibers of defined fiber spacing. In some cases, a subject 3-D filamentous fiber matrix comprises fibers that have a fiber spacing of about 5 μm, about 10 μm, about 15 μm, about 20 μm, about 25 μm, about 30 μm, about 35 μm, about 40 μm, about 45 μm, about 46 μm, about 47 μm, about 48 μm, about 49 μm, about 50 μm, about 51 μm, about 52 μm, about 53 μm, about 54 μm, about 55 μm, about 60 μm, about 65 μm, about 70 μm, about 75 μm, about 80 μm, about 85 μm, about 90 μm, about 95 μm, about 100 μm in the X-axis. In some cases, a subject 3-D filamentous fiber matrix comprises fibers that have a fiber spacing of 50 μm in the X-axis. Any suitable fiber spacing may be used according to the type of cells that are desired to be grown on subject filamentous matrices. A suitable fiber spacing may mimic the dimensions that are found in the cell type's native environment.

In some embodiments, a subject 3-D filamentous fiber matrix comprises fibers arranged in defined layer spacing in the Z-axis. In some cases, a subject 3-D filamentous fiber matrix comprises fibers arranged in layer spacing that can be about 1 μm, about 2 μm, about 5 μm, about 10 μm, about 15 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 45 μm, about 50 μm, about 55 μm, about 60 μm, about 65 μm, about 70 μm in the X-axis. In some cases, a subject 3-D filamentous fiber matrix comprises fibers arranged in layer spacing of 30 μm in the X-axis. Any suitable fiber length may be used according to the type of cells that are desired to be grown on subject filamentous matrices. A suitable layer spacing may mimic the dimensions that are found in the cell type's native environment.

In some embodiments, a subject 3-D filamentous fiber matrix comprises fibers of defined diameter. In some cases, a subject 3-D filamentous fiber matrix comprises fibers of diameter that can be about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm. In some cases, a subject 3-D filamentous fiber matrix comprises fibers that have a diameter of 5 μm. In some cases, a subject 3-D filamentous fiber matrix comprises fibers that have a diameter of 10 μm. Any suitable fiber diameter may be used according to the type of cells that are desired to be grown on subject filamentous matrices. A suitable fiber diameter may mimic, e.g., the dimensions that are found in the cell type's native environment, the rigidity of the cell type's native environment, the contractility of the cell type's native environment.

In some embodiments, multiple filamentous matrices are fabricated onto the same device (e.g., a glass slide; a multi-well cell culture plate; etc.). In some cases, 2 filamentous matrices are fabricated onto the same device (solid support). In some cases, 3, 4, 5, 6, 7, 8, 9, 10, 11, 12, 13, 14, 15, 16, 17, 18, 19, 20 or more filamentous matrices are fabricated onto the same device. Multiple filamentous matrices fabricated onto the same device are spaced apart by a defined matrix spacing (see, FIG. 8). In some cases, the matrix spacing is defined such that each 3-D filamentous fiber matrix is, e.g., about 0.1 mm apart, about 0.2 mm apart, about 0.3 mm apart, about 0.4 mm apart, about 0.5 mm apart, about 0.6 mm apart, about 0.7 mm apart, about 0.8 mm apart, about 0.9 mm apart, about 1.0 mm apart, about 1.1 mm apart, about 1.2 mm apart, about 1.3 mm apart, about 1.4 mm apart, about 1.5 mm apart, about 1.6 mm apart, about 1.7 mm apart, about 1.8 mm apart, about 1.9 mm apart, about 2.0 mm apart, about 2.1 mm apart, about 2.2 mm apart, about 2.3 mm apart, about 2.4 mm apart, about 2.5 mm apart, about 2.6 mm apart, about 2.7 mm apart, about 2.8 mm apart, about 2.9 mm apart, about 3.0 mm apart. In some cases, each 3-D filamentous fiber matrix is spaced 2.0 mm apart in the X-axis. A device comprising multiple filamentous matrices may increase the throughput in which structures, e.g., microtissues are cultured.

Cells

Cells that can be cultured on a 3-D filamentous fiber matrix of the present disclosure include stem cells; induced pluripotent stem (iPS) cells; human embryonic stem (hES) cells; mesenchymal stem cells (MSCs); multipotent progenitor cells; cardiomyocytes; cardiomyocyte progenitors; hepatocytes; beta islet cells; neurons, e.g., astrocytes, neuronal sub-populations; leukocytes; endothelial cells; lung epithelial cells; exocrine secretory epithelial cells; hormone-secreting cells, such as anterior pituitary cells, magnocellular neurosecretory cells, thyroid epithelial cells, adrenal gland cells, etc.; keratinocytes; lymphocytes; macrophages; monocytes; renal cells; urethral cells; sensory transducer cells; autonomic neuronal cells; central nervous system neurons; glial cells; skeletal muscle cells; a kidney cell, e.g., a kidney parietal cell, a kidney glomerulus podocyte, etc.; white adipocytes (e.g., white adipose tissue (WAT)), brown adipocytes; adipose-derived stem cells; osteocytes; osteoblasts; chondrocytes; smooth muscle cells; microglial cells; stromal cells; etc. In some embodiments, a cell is genetically modified to express a reporter polypeptide.

In some embodiments, stem cells or progenitor cells that have been differentiated into cells of one or more specific organs or tissues are cultured on a 3-D filamentous fiber matrix. In certain embodiments, a stem cell or progenitor cell is initially cultured in a subject 3-D filamentous fiber matrix, and the stem cell or progenitor cell is then differentiated into a specific cell type.

In some cases, cells cultured in a 3-D filamentous fiber matrix of the present disclosure are healthy. In some cases, cells cultured in a 3-D filamentous fiber matrix of the present disclosure are diseased. In some cases, cells cultured in a 3-D filamentous fiber matrix of the present disclosure include one or more genetic mutations that pre dispose the cells to disease. Both non-cancerous as well as cancerous cells can be cultured in the subject 3-D filamentous fiber matrix. In some embodiments, cells from a cancer cell line are cultured in the subject 3-D filamentous fiber matrix. In certain embodiments, cells from a breast cancer cell line are cultured in the subject 3-D filamentous fiber matrix.

In some cases, the cells cultured in a 3-D filamentous fiber matrix of the present disclosure are primary cells. In some cases, the cells cultured in a 3-D filamentous fiber matrix of the present disclosure are primary cells obtained from a healthy individual. In some cases, the cells cultured in a 3-D filamentous fiber matrix of the present disclosure are primary cells obtained from a diseased individual. In some cases, the cells cultured in a 3-D filamentous fiber matrix of the present disclosure are obtained from an individual who has a disease-associated mutation, but who has not been diagnosed as having a disease associated with the disease-associated mutation. In some cases, the cells cultured in a 3-D filamentous fiber matrix of the present disclosure are all obtained from a single individual. In some cases, the cells cultured in a 3-D filamentous fiber matrix of the present disclosure are obtained from two or more different individuals.

In some cases, the cells cultured in a 3-D filamentous fiber matrix of the present disclosure are human cells. In some cases, the cells cultured in a 3-D filamentous fiber matrix of the present disclosure are non-human mammalian cells. In some cases, the cells cultured in a 3-D filamentous fiber matrix of the present disclosure are rat cells. In some cases, the cells cultured in a 3-D filamentous fiber matrix of the present disclosure are mouse cells. In some cases, the cells cultured in a 3-D filamentous fiber matrix of the present disclosure are pig cells. In some cases, the cells cultured in a 3-D filamentous fiber matrix of the present disclosure are non-human primate cells.

Cardiomyocytes

In some cases, cells that are cultured in a 3-D filamentous fiber matrix of the present disclosure are cardiomyocytes. The following discussion as it relates to cardiomyocytes is applicable to any of a variety of cell types, as described above, which may be cultured in a subject mi 3-D filamentous fiber matrix. The following discussion of cardiomyocytes is therefore exemplary and not intended to be limiting.

Cells that can be cultured in a 3-D filamentous fiber matrix of the present disclosure include cardiomyocytes, cardiomyocyte progenitors, induced pluripotent stem (iPS) cells, and the like. In some cases, the cardiomyocytes or cardiomyocyte progenitors are healthy cardiomyocytes or cardiomyocyte progenitors. In some cases, the cardiomyocytes or cardiomyocyte progenitors are diseased cardiomyocytes or cardiomyocyte progenitors. For example, in some cases, the cardiomyocytes or cardiomyocyte progenitors are from an individual having a cardiovascular disease or condition. For example, in some cases, the cardiomyocytes or cardiomyocyte progenitors are from an individual having an ischemic heart disease, an arrhythmia, tachycardia, bradycardia, myocardial infarction, or a congenital heart condition. For example, in some cases, the cardiomyocytes or cardiomyocyte progenitors are from an individual having long QT syndrome (LQTS). Congenital LQTS is an inherited cardiac arrhythmic disease that results from ion channel defects. Drug-induced LQTS can be acquired following use of certain pharmaceutical agents. In some embodiments, human cardiac myocyte cells are cultured in the subject 3-D filamentous fiber matrix. In some embodiments, dilated cardiomyopathy (DCM) cells are cultured in the subject 3-D filamentous fiber matrix. In some embodiments, hypertrophic cardiomyopathy (HCM) cells are cultured in the subject 3-D filamentous fiber matrix. In some embodiments, cells cultured in a 3-D filamentous fiber matrix of the present disclosure may be obtained from individuals having severe DCM phenotypes and childhood early death. In some cases, cells cultured in a 3-D filamentous fiber matrix of the present disclosure may be obtained from individuals having adult-onset HCM, that results in genetic predisposition for heart failure with risk increased by hypertension, age, and other environmental factors.

Cells that can be cultured in a 3-D filamentous fiber matrix of the present disclosure include induced pluripotent stem cells (iPS cells). In some embodiments, human iPS cardiomyocytes (hiPS-CMs) are cultured in a 3-D filamentous fiber matrix of the present disclosure. In some cases, the iPS cells are generated from somatic cells obtained from healthy individuals. In some cases, the iPS cells are generated from somatic cells obtained from individuals having a cardiovascular disease or condition. For example, in some cases, the iPS cells are generated from a somatic cell obtained from an individual having a cardiovascular disease or condition such as ischemic heart disease, arrhythmia, tachycardia, bradycardia, myocardial infarction, hypertrophic cardiomyopathy (HCM), dilated cardiomyopathy (DCM) or a congenital heart condition. In some cases, the iPS cells are generated from somatic cells obtained from individuals having severe DCM phenotypes and childhood early death. In some cases, the iPS cells are generated from somatic cells obtained from individuals having adult-onset HCM, that results in genetic predisposition for heart failure with risk increased by hypertension, age, and other environmental factors.

Cardiomyocytes can have certain morphological characteristics. They can be spindle, round, triangular or multi-angular shaped, and they may show striations characteristic of sarcomeric structures detectable by immunostaining. They may form flattened sheets of cells, or aggregates that stay attached to the substrate or float in suspension, showing typical sarcomeres and atrial granules when examined by electron microscopy

Cardiomyocytes and cardiomyocyte precursors generally express one or more cardiomyocyte-specific markers. Cardiomyocyte-specific markers include, but are not limited to, cardiac troponin I (cTnI), cardiac troponin-C, cardiac troponin T (cTnT), tropomyosin, caveolin-3, myosin heavy chain (MHC), myosin light chain-2a, myosin light chain-2v, ryanodine receptor, sarcomeric α-actinin, Nkx2.5, connexin 43, and atrial natriuretic factor (ANF). Cardiomyocytes can also exhibit sarcomeric structures. Cardiomyocytes exhibit increased expression of cardiomyocyte-specific genes ACTC1 (cardiac α-actin), ACTN2 (actinin a2), MYH6 (α-myosin heavy chain), RYR2 (ryanodine receptor 2), MYL2 (myosin regulatory light chain 2, ventricular isoform), MYL7 (myosin regulatory light chain, atrial isoform), TNNT2 (troponin T type 2, cardiac), and NPPA (natriuretic peptide precursor type A), PLN (phospholamban).

In some cases, cardiomyocytes can express cTnI, cTnT, Nkx2.5; and can also express at least 3, 4, 5, or more than 5, of the following: ANF, MHC, titin, tropomyosin, α-sarcomeric actinin, desmin, GATA-4, MEF-2A, MEF-2B, MEF-2C, MEF-2D, N-cadherin, connexin-43, β-1-adrenoreceptor, creatine kinase MB, myoglobin, α-cardiac actin, early growth response-I, and cyclin D2.

In some cases, a cardiomyocyte is generated from an iPS cell, where the iPS cell is generated from a somatic cell obtained from an individual.

Patient-Specific Cells

In some cases, the cells are patient-specific cells. In some cases, the patient-specific cells are derived from stem cells obtained from a patient. In some cases, the patient-specific cells are derived from iPS cells generated from somatic cells obtained from a patient. In some cases, patient-specific cells are primary cells. In some cases, the cells form embryoid bodies (EBs).

Suitable stem cells include embryonic stem cells, adult stem cells, and induced pluripotent stem (iPS) cells.

iPS cells are generated from mammalian cells (including mammalian somatic cells) using, e.g., known methods. Examples of suitable mammalian cells include, but are not limited to: fibroblasts, skin fibroblasts, dermal fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, epithelial cells, gastric epithelial cells, lung epithelial cells, synovial cells, kidney cells, skin epithelial cells, pancreatic beta cells, and osteoblasts.

Mammalian cells used to generate iPS cells can originate from a variety of types of tissue including but not limited to: bone marrow, skin (e.g., dermis, epidermis), muscle, adipose tissue, peripheral blood, foreskin, skeletal muscle, and smooth muscle. The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

iPS cells produce and express on their cell surface one or more of the following cell surface antigens: SSEA-3, SSEA-4, TRA-1-60, TRA-1-81, TRA-2-49/6E (alkaline phophatase), and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT.

Methods of generating iPS cells are known in the art, and a wide range of methods can be used to generate iPS cells. See, e.g., Takahashi and Yamanaka (2006) Cell 126:663-676; Yamanaka et al. (2007) Nature 448:313-7; Wernig et al. (2007) Nature 448:318-24; Maherali (2007) Cell Stem Cell 1:55-70; Maherali and Hochedlinger (2008) Cell Stem Cell 3:595-605; Park et al. (2008) Cell 134:1-10; Dimos et. al. (2008) Science 321:1218-1221; Blelloch et al. (2007) Cell Stem Cell 1:245-247; Stadtfeld et al. (2008) Science 322:945-949; Stadtfeld et al. (2008) 2:230-240; Okita et al. (2008) Science 322:949-953.

In some embodiments, iPS cells are generated from somatic cells by forcing expression of a set of factors in order to promote increased potency of a cell or de differentiation. Forcing expression can include introducing expression vectors encoding polypeptides of interest into cells, introducing exogenous purified polypeptides of interest into cells, or contacting cells with a reagent that induces expression of an endogenous gene encoding a polypeptide of interest.

Forcing expression may include introducing expression vectors into somatic cells via use of moloney-based retroviruses (e.g., MLV), lentiviruses (e.g., HIV), adenoviruses, protein transduction, transient transfection, or protein transduction. In some embodiments, the moloney-based retroviruses or HIV-based lentiviruses are pseudotyped with envelope from another virus, e.g. vesicular stomatitis virus g (VSV-g) using known methods in the art. See, e.g. Dimos et al. (2008) Science 321:1218-1221.

In some embodiments, iPS cells are generated from somatic cells by forcing expression of Oct-3/4 and Sox2 polypeptides. In some embodiments, iPS cells are generated from somatic cells by forcing expression of Oct-3/4, Sox2 and Klf4 polypeptides. In some embodiments, iPS cells are generated from somatic cells by forcing expression of Oct-3/4, Sox2, Klf4 and c-Myc polypeptides. In some embodiments, iPS cells are generated from somatic cells by forcing expression of Oct-4, Sox2, Nanog, and LIN28 polypeptides.

For example, iPS cells can be generated from somatic cells by genetically modifying the somatic cells with one or more expression constructs encoding Oct-3/4 and Sox2. As another example, iPS cells can be generated from somatic cells by genetically modifying the somatic cells with one or more expression constructs comprising nucleotide sequences encoding Oct-3/4, Sox2, c-myc, and Klf4. As another example, iPS cells can be generated from somatic cells by genetically modifying the somatic cells with one or more expression constructs comprising nucleotide sequences encoding Oct-4, Sox2, Nanog, and LIN28.

In some embodiments, cells undergoing induction of pluripotency as described above, to generate iPS cells, are contacted with additional factors which can be added to the culture system, e.g., included as additives in the culture medium. Examples of such additional factors include, but are not limited to: histone deacetylase (HDAC) inhibitors, see, e.g. Huangfu et al. (2008) Nature Biotechnol. 26:795-797; Huangfu et al. (2008) Nature Biotechnol. 26: 1269-1275; DNA demethylating agents, see, e.g., Mikkelson et al (2008) Nature 454, 49-55; histone methyltransferase inhibitors, see, e.g., Shi et al. (2008) Cell Stem Cell 2:525-528; L-type calcium channel agonists, see, e.g., Shi et al. (2008) 3:568-574; Wnt3a, see, e.g., Marson et al. (2008) Cell 134:521-533; and siRNA, see, e.g., Zhao et al. (2008) Cell Stem Cell 3: 475-479.

In some embodiments, iPS cells are generated from somatic cells by forcing expression of Oct3/4, Sox2 and contacting the cells with an HDAC inhibitor, e.g., valproic acid. See, e.g., Huangfu et al. (2008) Nature Biotechnol. 26: 1269-1275. In some embodiments, iPS cells are generated from somatic cells by forcing expression of Oct3/4, Sox2, and Klf4 and contacting the cells with an HDAC inhibitor, e.g., valproic acid. See, e.g., Huangfu et al. (2008) Nature Biotechnol. 26:795-797.

Cardiomyocytes (e.g., patient-specific cardiomyocytes) can be generated from iPS cells using any known method. See, e.g., Mummery et al. (2012) Circ. Res. 111:344.

Under appropriate circumstances, iPS cell-derived cardiomyocytes often show spontaneous periodic contractile activity. This means that when they are cultured in a suitable tissue culture environment with an appropriate Ca²⁺ concentration and electrolyte balance, the cells can be observed to contract across one axis of the cell, and then release from contraction, without having to add any additional components to the culture medium. The contractions are periodic, which means that they repeat on a regular or irregular basis, at a frequency between about 6 and 200 contractions per minute, and often between about 20 and about 90 contractions per minute in normal buffer. Individual cells may show spontaneous periodic contractile activity on their own, or they may show spontaneous periodic contractile activity in concert with neighboring cells in a tissue, cell aggregate, or cultured cell mass.

Generation of Cardiomyocytes from iPSCs

Cardiomyocytes can be generated from iPSCs, or other stem cells, using well-known methods/See, e.g., Mummery et al. (2012) Circ. Res. 111:344; Lian et al. (2012) Proc. Natl. Acad. Sci. USA 109:E1848; Ye et al. (2013) PLoS One 8:e53764.

Generation of Cardiomyocytes Directly from a Post-Natal Somatic Cell

A cardiomyocyte can be generated directly from a post-natal somatic cell, without formation of an iPS cell as an intermediate. For example, in some cases, a human post-natal fibroblast is induced directly (to become a cardiomyocyte, using a method as described in WO 2014/033123. For example, reprogramming factors Gata4, Mef2c, Tbx5, Mesp1, and Essrg are introduced into a human post-natal fibroblast to induce the human post-natal fibroblast to become a cardiomyocyte. In some cases, the polypeptides themselves are introduced into the post-natal fibroblast. In other cases, the post-natal fibroblast is genetically modified with one or more nucleic acids comprising nucleotide sequences encoding Gata4, Mef2c, Tbx5, Mesp1, and Essrg.

Isogenic Pairs of Cardiomyocytes

In some cases, isogenic pairs of cardiomyocytes are used. In some cases, isogenic pairs of wild-type and genetically modified cardiomyocytes are used. In some cases, isogenic pairs of diseased and non-diseased cardiomyocytes are used. For example, in some cases, isogenic pairs of cardiomyocytes from an individual are used, where one of the isogenic pair is genetically modified with a nucleic acid comprising a nucleotide sequence encoding a mutant form of a polypeptide such that the genetically modified cardiomyocyte exhibits characteristics of a diseased cardiomyocyte.

In some cases, isogenic pairs of iPS cells are used. In some cases, isogenic pairs of wild-type and genetically modified iPS cells are used. In some cases, isogenic pairs of diseased and non-diseased iPS cells are used.

In some cases, isogenic homozygous null human iPS cells are used. For example, in some cases, isogenic homozygous MYBPC3 null human iPS cells are used. MYBPC3 is a thick filament associated protein, which is thought to play a principally structural role stabilization of the sarcomere sliding during contraction. Isogenic homozygous human iPS cells null for any gene of interest may be used. In some cases, null human iPS cells are generated by TALEN-mediated gene editing methods. Any known gene editing methods can be used, e.g., meganuclease-mediated gene editing methods, zinc finger nuclease-mediated gene editing methods, CRISPR-Cas mediated gene editing methods.

Genetic Modification

In some cases, a cell cultured in a subject 3-D filamentous fiber matrix is genetically modified. For example, a cell can be genetically altered to express one or more growth factors of various types, such as FGF, cardiotropic factors such as atrial natriuretic factor, cripto, and cardiac transcription regulation factors, such as GATA-4, Nkx2.5, and MEF2-C. Genetic modification generally involves introducing into the cell a nucleic acid comprising a nucleotide sequence encoding a polypeptide of interest. The nucleotide sequence encoding the polypeptide of interest can be operably linked to a transcriptional control element, such as a promoter. Suitable promoters include, e.g., promoters of cardiac troponin I (cTnI), cardiac troponin T (cTnT), sarcomeric myosin heavy chain (MHC), GATA-4, Nkx2.5, N-cadherin, .beta.1-adrenoceptor, ANF, the MEF-2 family of transcription factors, creatine kinase MB (CK-MB), myoglobin, or atrial natriuretic factor (ANF).

In some cases, a cardiomyocyte is genetically modified with a nucleic acid comprising a nucleotide sequence encoding a mutant form of a polypeptide such that the genetically modified cardiomyocyte exhibits characteristics of a diseased cardiomyocyte. For example, a cardiomyocyte can be genetically modified to express a KVLQT1, HERG, SCN5A, KCNE1, or KCNE2 polypeptide comprising a mutation associated with LQTS, where the genetically modified cardiomyocyte exhibits characteristics associated with LQTS. See, e.g., Splawski et al. (2000) Circulation 102:1178, for mutations in KVLQT1, HERG, SCN5A, KCNE1, and KCNE2 that are associated with LQTS. For example, a cardiomyocyte can be genetically modified such that a gene encoding a KVLQT1, HERG, SCN5A, KCNE1, or KCNE2 polypeptide with a LQTS-associated mutation replaces a wild-type KVLQT1, HERG, SCN5A, KCNE1, or KCNE2 gene.

In some cases, a cell to be cultured in a subject 3-D filamentous fiber matrix is genetically modified to express one or more polypeptides that provide real-time detection of a cellular response. Such polypeptides include, e.g., calcium indicators, genetically encoded voltage indicators (GEVI; e.g., voltage-sensitive fluorescent proteins), sodium channel protein activity indicators, indicators of oxidation/reduction status within the cell, etc. For example, a cell can be genetically modified to include an indicator of Cyp3A4 activity.

In some cases, a cell (e.g., a cardiomyocyte or other cell) is genetically modified to express a genetically-encoded calcium indicator (GECI). See, e.g., Mank and Griesbeck (2008) Chem. Rev. 108:1550; Nakai et al. (2001) Nat. Biotechnol. 19:137; Akerboom et al. (2012) J. Neurosci. 32:13819; Akerboom et al. (2013) Front. Mol. Neurosci. 6:2. Suitable GECI include pericams, cameleons (Miyawaki et al (1999) Proc. Natl. Acad. Sci. USA 96:2135), and GCaMP. As one non-limiting example, a suitable GECI can be a fusion of a circularly permuted variant of enhanced green fluorescent protein (cpEGFP) with the calcium-binding protein calmodulin (CaM) at the C terminus and a CaM-binding M13 peptide (from myosin light chain) at the N terminus. Nakai et al. (2001) Nat. Biotechnol. 19:137. In some cases, a suitable GECI can comprise an amino acid sequence having at least 85%, at least 90%, at least 95%, at least 98%, or 100%, amino acid sequence identity with the following GCaMP6 amino acid sequence:

(SEQ ID NO: 1)
MGSHHHHHHG MASMTGGQQM GRDLYDDDDK DLATMVDSSR
RKWNKTGHAV RAIGRLSSLE NVYIKADKQK NGIKANFKIR
HNIEDGGVQL AYHYQQNTPI GDGPVLLPDN HYLSVQSKLS
KDPNEKRDHM VLLEFVTAAG ITLGMDELYK GGTGGSMVSK
GEELFTGVVP ILVELDGDVN GHKFSVSGEG EGDATYGKLT
LKFICTTGKL PVPWPTLVTT LXVQCFSRYP DHMKQHDFFK
SAMPEGYIQE RTIFFKDDGN YKTRAEVKFE GDTLVNRIEL
KGIDFKEDGN ILGHKLEYNL PDQLTEEQIA EFKEAFSLFD
KDGDGTITTK ELGTVMRSLG QNPTEAELQD MINEVDADGD
GTIDFPEFLT MMARKGSYRD TEEEIREAFG VFDKDGNGYI
SAAELRHVMT NLGEKLTDEE VDEMIREADI DGDGQVNYEE
FVQMMTAK

In some cases, the GECI is GCaMP6f.

Systems

The present disclosure provides a system comprising a 3-D filamentous fiber matrix of the present disclosure.

In some cases, a system of the present disclosure comprises: a) a first three-dimensional filamentous fiber matrix comprising a first cell population comprising a mutation in a gene encoding a gene product required for normal cellular function, wherein the mutation reduces the level or the activity of the gene product; and b) a second three-dimensional filamentous fiber matrix comprising a second cell population, wherein the second cell population is isogenic with the first cell population, but does not comprise the mutation, where the first and the second matrices are present on a solid support and separated from one another by a distance of from 1 mm to 5 mm.

In some cases, a system of the present disclosure comprises: a) a first three-dimensional filamentous fiber matrix comprising a first cardiomyocyte population comprising a mutation in a gene encoding a gene product required for normal cardiomyocyte function, wherein the mutation reduces the level or the activity of the gene product; and b) a first three-dimensional filamentous fiber matrix comprising a second cardiomyocyte population, wherein the second cardiomyocyte population is isogenic with the first cardiomyocyte population, but does not comprise the mutation, wherein the first and the second matrices are present on a solid support and separated from one another by a distance of from 1 mm to 5 mm.

Gene products whose level or activity can be affected by the mutation include, e.g., sarcomeric polypeptides, desmosome polypeptides, cytoskeletal polypeptides, Z-disk polypeptides, ion channel polypeptides, and the like. For example, in some cases, the gene product is a cardiac myosin binding protein C polypeptide. In some cases, the mutation is in a titin (TTN) gene. Other genes include genes encoding cytoskeletal (S-sarcoglycan (SGCD), β-sarcoglycan (SGCB), desmin (DES), lamin A/C (LMNA), vinculin (VCL)), sarcomeric/myofibrillar (α-cardiac actin (ACTC), troponin T (TNNT2), troponin I (TNNI3), β-myosin heavy chain (MYH7), myosin binding protein C (MBPC3), and α-tropomyosin (TPM1)), and Z-disk proteins (muscle LIM protein (MLP)/cysteine and glycine-rich protein 3 (CSRP3), titin (TTN), telethonin/TCAP, α-actinin-2 (ACTN2), nebulette (NEBL), myopalladin (MYPN), ANKRD1/CARP, and ZASP/LIM-domain binding 3 (LBD3). Other genes of interest include genes encoding cardiac sodium channel gene SCN5A and calcium homeostasis regulator phospholamban (PLN). Other genes of interest include genes encoding desmosome polypeptides, including, e.g., desmoplakin (DSP), desmoglein-2 (DSG2), and desmocolin-2 (DSC2).

In some cases, the mutation is a loss-of-function mutation. The mutation can be a homozygous mutation or a heterozygous mutation.

The cells present in the system can be derived from any of a number of sources. The cells can be human cells, non-human primate cells, rodent cells, ungulate cells, canine cells, equine cells, etc. The cells in many cases are mammalian cells. The cells can be primary cells, e.g., primary cells obtained from a mammal. The cells can be induced from iPS cells generated from primary cells obtained from a mammal.

In some cases, the cells are genetically modified to produce a polypeptide calcium reporter. For example, a cardiomyocyte can be genetically modified to produce a polypeptide calcium reporter, for ease of monitoring calcium flux. In some cases, the calcium reporter is GCaMP6f.

A system of the present disclosure can comprise, in addition to a 3-D filamentous fiber matrix of the present disclosure, one or more devices for measuring various cell parameters. In some cases, the device is capable of tracking motion of cells in the matrix (e.g., cardiomyocytes in the matrix). The Examples provide a description an exemplary device for tracking motion of cells. In some cases, the device is capable of measuring deflection of the filamentous fibers in the matrices in response to cardiomyocyte contraction. Measuring deflection of the filamentous fibers in the matrix provides a measure of the force exerted on the fiber by a cardiomyocyte (or cardiac microtis sue) upon contraction. The Examples provide a description of measuring deflection of filamentous fibers in a matrix of the present disclosure.

2. The matrix of claim 1, wherein the gene product is selected from a cardiac myosin binding protein C polypeptide, a cytoskeletal polypeptide, δ-sarcoglycan (SGCD), β-sarcoglycan (SGCB), desmin (DES), lamin A/C (LMNA), vinculin (VCL), a sarcomeric/myofibrillar polypeptide, α-cardiac actin (ACTC), troponin T (TNNT2), troponin I (TNNI3), β-myosin heavy chain (MYH7), myosin binding protein C (MBPC3), α-tropomyosin (TPM1), a Z-disk protein, muscle LIM protein (MLP), cysteine and glycine-rich protein 3 (CSRP3), titin (TTN), telethonin/TCAP, α-actinin-2 (ACTN2), nebulette (NEBL), myopalladin (MYPN), ANKRD1/CARP, ZASP/LIM-domain binding 3 (LBD3), cardiac sodium channel gene SCN5A, calcium homeostasis regulator phospholamban (PLN), desmoplakin (DSP), desmoglein-2 (DSG2), and desmocolin-2 (DSC2).

In some cases, as described above, the first and the second matrix comprise filamentous fibers having a diameter of from 2 μm to 20 μm. In some cases, as described above, the first and the second matrix comprise comprises filamentous fibers having a diameter of from 5 μm to 10 μm. In some cases, as described above, the first and the second matrix comprise filamentous fibers, each fiber comprising a first end and a second end, wherein the first end and the second end of the fiber are attached to a solid support. In some cases, as described above, the solid support comprises glass or a non-water-soluble polymer (water insoluble polymer). In some cases, as described above, the filamentous fibers are from 450 μm to 600 μm in length in the Y-axis. In some cases, as described above, the filamentous fibers form layers spaced from about 40 μm to about 60 μm apart in the X-axis, and wherein the layers are spaced from about 25 μm to about 35 μm in the Z-axis. In some cases, as described above, the filamentous fibers have an elastic modulus of from about 160 MPa to about 200 MPa. In some cases, as described above, the filamentous fibers have an elastic modulus of from about 170 MPa to about 190 MPa. In some cases, as described above, the cardiomyocytes are present in the matrices at a density of from 1×10⁶ cells/cc to 6×10⁶ cells/cc. In some cases, as described above, the cardiomyocytes are present in the matrices at a density of from 2×10⁶ cells/cc to 5×10⁶ cells/cc.

Methods

A 3-D filamentous fiber matrix of the present disclosure, and a system of the present disclosure, are useful in various applications. Such applications include, e.g., characterizing a mutation (e.g., a previously unknown mutation) in a gene encoding a gene product such as a sarcomeric gene; identifying a candidate agent for treating a cardiomyopathy; and the like.

Characterizing a Mutation

The present disclosure provides a method of characterizing a mutation in a gene encoding a gene product required for normal cardiomyocyte function, the method comprising measuring deflection of the filamentous fibers in the matrices in response to cardiomyocyte contraction in a matrix of the present disclosure, wherein the cardiomyocytes comprising a mutation in a gene encoding a gene product required for normal cardiomyocyte function, wherein the mutation reduces the level or the activity of the gene product. In some cases, the method comprises a control, e.g., an isogenic cardiomyocyte that does not include the mutation. Comparison of the deflection of the filamentous fibers in the matrices in response to cardiomyocyte contraction by the mutated cardiomyocyte is compared to the deflection of the filamentous fibers in the matrices in response to cardiomyocyte contraction by the isogenic cardiomyocyte that does not include the mutation. Where the deflection generated by the mutated cardiomyocyte is reduced relative to that generated by the non-mutated isogenic cardiomyocyte, the mutation can be considered to affect contraction.

Screening Methods

The present disclosure provides a method of identifying a candidate agent for treating a cardiomyopathy, the method comprising: a) contacting cardiomyocytes in a matrix of the present disclosure with a test agent, wherein the cardiomyocytes comprising a mutation in a gene encoding a gene product required for normal cardiomyocyte function, wherein the mutation reduces the level or the activity of the gene product; and b) measuring the effect of the test agent on deflection of the filamentous fibers in the matrix in response to cardiomyocyte contraction, wherein a test agent that increases the deflection, compared to a control, is a candidate agent for treating a myopathy. In some cases, a test agent that increases the deflection by at least 5%, at least 10%, at least 15%, at least 20%, at least 25%, at least 30%, at least 35%, at least 40%, at least 50%, or more than 50%, compared to a control, is a candidate agent for treating a myopathy.

In some cases, the the cardiomyocytes are obtained from an individual with a cardiomyopathy. In some cases, the cardiomyocytes are generated from induced pluripotent stem cells generated from cells obtained from an individual with a cardiomyopathy.

The term “test agent” as used herein describes any molecule, e.g., ion, inorganic oxyanion, metal oxyanion, organic small molecule, secondary metabolite, peptide, lipid, carbohydrate, polynucleotide, protein, drug or pharmaceutical. Generally, a plurality of assay mixtures is run in parallel with different agents or agent concentrations to obtain a differential response to the various agents or agent concentrations. In some cases, one of these samples serves as a negative control, e.g., at zero concentration or below the level of detection.

Compounds of interest for screening include biologically active agents of numerous chemical classes, primarily organic molecules, which may include organometallic molecules, inorganic molecules, etc. Test agents can encompass numerous chemical classes, such as organic molecules, e.g., small organic compounds having a molecular weight of more than 50 and less than about 2,500 daltons. A test agent can have a molecular weight greater than 2,500 daltons, e.g., from 2.5 kDa to about 50 kDa. Test agents can comprise functional groups necessary for structural interaction with proteins, particularly hydrogen bonding, and may include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Of interest in certain embodiments are compounds that pass cellular membranes.

Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-39 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A three-dimensional filamentous fiber matrix comprising:

a) a first cardiomyocyte population comprising a mutation in a gene encoding a gene product required for normal cardiomyocyte function, wherein the mutation reduces the level or the activity of the gene product; and/or

b) a second cardiomyocyte population, wherein the second cardiomyocyte population is isogenic with the first cardiomyocyte population, but does not comprise the mutation.

Aspect 2. The matrix of aspect 1, wherein the gene product is selected from a cardiac myosin binding protein C polypeptide, a cytoskeletal polypeptide, δ-sarcoglycan (SGCD), β-sarcoglycan (SGCB), desmin (DES), lamin A/C (LMNA), vinculin (VCL), a sarcomeric/myofibrillar polypeptide, α-cardiac actin (ACTC), troponin T (TNNT2), troponin I (TNNI3), β-myosin heavy chain (MYH7), myosin binding protein C (MBPC3), α-tropomyosin (TPM1), a Z-disk protein, muscle LIM protein (MLP), cysteine and glycine-rich protein 3 (CSRP3), titin (TTN), telethonin/TCAP, α-actinin-2 (ACTN2), nebulette (NEBL), myopalladin (MYPN), ANKRD1/CARP, ZASP/LIM-domain binding 3 (LBD3), cardiac sodium channel gene SCN5A, calcium homeostasis regulator phospholamban (PLN), desmoplakin (DSP), desmoglein-2 (DSG2), and desmocolin-2 (DSC2).

Aspect 3. The matrix of aspect 1, wherein the mutation is a loss-of-function mutation.

Aspect 4. The matrix of aspect 1, wherein the first and the second cardiomyocyte populations are human cardiomyocytes.

Aspect 5. The matrix of aspect 1, wherein the first cardiomyocyte population is genetically modified to produce a polypeptide calcium reporter.

Aspect 6. The matrix of aspect 5, wherein the calcium reporter is GCaMP6f.

Aspect 7. The matrix of any one of aspects 1-6, wherein the matrix comprises filamentous fibers having a diameter of from 2 μm to 20 μm.

Aspect 8. The matrix of any one of aspects 1-6, wherein the matrix comprises filamentous fibers having a diameter of from 5 μm to 10 μm.

Aspect 9. The matrix of any one of aspects 1-8, wherein the matrix comprises filamentous fibers, each fiber comprising a first end and a second end, wherein the first end and the second end of the fiber are attached to a solid support.

Aspect 10. The matrix of aspect 9, wherein the solid support comprises glass or a non-water-soluble polymer.

Aspect 11. The matrix of any one of aspects 1-10, wherein the filamentous fibers are from 450 μm to 600 μm in length in the Y-axis.

Aspect 12. The matrix of any one of aspects 1-11, wherein the filamentous fibers form layers spaced from about 40 μm to about 60 μm apart in the X-axis, and wherein the layers are spaced from about 25 μm to about 35 μm in the Z-axis.

Aspect 13. The matrix of any one of aspects 1-12, wherein the filamentous fibers have an elastic modulus of from about 160 MPa to about 200 MPa.

Aspect 14. The matrix of any one of aspects 1-12, wherein the filamentous fibers have an elastic modulus of from about 170 MPa to about 190 MPa.

Aspect 15. The matrix of any one of aspects 1-14, wherein the cardiomyocytes are present in the matrix at a density of from 1×10⁶ cells/cc to 6×10⁶ cells/cc.

Aspect 16. The matrix of any one of aspects 1-14, wherein the cardiomyocytes are present in the matrix at a density of from 2×10⁶ cells/cc to 5×10⁶ cells/cc.

Aspect 17. A system comprising:

a) a first three-dimensional filamentous fiber matrix comprising a first cardiomyocyte population comprising a mutation in a gene encoding a gene product required for normal cardiomyocyte function, wherein the mutation reduces the level or the activity of the gene product; and

b) a second three-dimensional filamentous fiber matrix comprising a second cardiomyocyte population, wherein the second cardiomyocyte population is isogenic with the first cardiomyocyte population, but does not comprise the mutation, wherein the first and the second matrices are present on a solid support and separated from one another by a distance of from 1 mm to 5 mm.

Aspect 18. The system of aspect 17, wherein the gene product is a cardiac myosin binding protein C polypeptide.

Aspect 19. The system of aspect 17, wherein the mutation is a loss-of-function mutation.

Aspect 20. The system of aspect 17, wherein the first and the second cardiomyocyte populations are human cardiomyocytes.

Aspect 21. The system of aspect 17, wherein the first cardiomyocyte population is genetically modified to produce a polypeptide calcium reporter.

Aspect 22. The system of aspect 21, wherein the calcium reporter is GCaMP6f.

Aspect 23. The system of any one of aspects 17-22, wherein the first and the second matrix comprises filamentous fibers having a diameter of from 2 μm to 20 μm.

Aspect 24. The system of any one of aspects 17-22, wherein the first and the second matrix comprises filamentous fibers having a diameter of from 5 μm to 10 μm.

Aspect 25. The system of any one of aspects 17-24, wherein the first and the second matrix comprises filamentous fibers, each fiber comprising a first end and a second end, wherein the first end and the second end of the fiber are attached to the solid support.

Aspect 26. The system of aspect 25, wherein the solid support comprises glass or a non-water-soluble polymer.

Aspect 27. The system of any one of aspects 17-26, wherein the filamentous fibers are from 450 μm to 600 μm in length in the Y-axis.

Aspect 28. The system of any one of aspects 17-27, wherein the filamentous fibers form layers spaced from about 40 μm to about 60 μm apart in the X-axis, and wherein the layers are spaced from about 25 μm to about 35 μm in the Z-axis.

Aspect 29. The system of any one of aspects 17-28, wherein the filamentous fibers have an elastic modulus of from about 160 MPa to about 200 MPa.

Aspect 30. The system of any one of aspects 17-28, wherein the filamentous fibers have an elastic modulus of from about 170 MPa to about 190 MPa.

Aspect 31. The system of any one of aspects 17-30, wherein the cardiomyocytes are present in the first and the second matrix at a density of from 1×10⁶ cells/cc to 6×10⁶ cells/cc.

Aspect 32. The system of any one of aspects 17-30, wherein the cardiomyocytes are present in the first and the second matrix at a density of from 2×10⁶ cells/cc to 5×10⁶ cells/cc.

Aspect 33. The system of any one of aspects 17-32, comprising a device for tracking motion of the cardiomyocytes.

Aspect 34. The system of any one of aspects 17-33, comprising a device for measuring deflection of the filamentous fibers in the matrices in response to cardiomyocyte contraction.

Aspect 35. The system of any one of aspects 17-34, comprising a device for measuring force applied by the cardiomyocytes on the filamentous fibers.

Aspect 36. A method of characterizing a mutation in a gene encoding a gene product required for normal cardiomyocyte function, the method comprising measuring deflection of the filamentous fibers in the matrices in response to cardiomyocyte contraction in a matrix of any one of aspects 1-16, wherein the cardiomyocytes comprising a mutation in a gene encoding a gene product required for normal cardiomyocyte function, wherein the mutation reduces the level or the activity of the gene product.

Aspect 37. A method of identifying a candidate agent for treating a cardiomyopathy, the method comprising:

a) contacting cardiomyocytes in a matrix of any one of aspects 1-16 with a test agent, wherein the cardiomyocytes comprising a mutation in a gene encoding a gene product required for normal cardiomyocyte function, wherein the mutation reduces the level or the activity of the gene product; and

b) measuring the effect of the test agent on deflection of the filamentous fibers in the matrix in response to cardiomyocyte contraction, wherein a test agent that increases the deflection, compared to a control, is a candidate agent for treating a myopathy.

Aspect 38. The method of aspect 37, wherein the cardiomyocytes are obtained from an individual with a cardiomyopathy.

Aspect 39. The method of aspect 37, wherein the cardiomyocytes are generated from induced pluripotent stem cells generated from cells obtained from an individual with a cardiomyopathy.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Materials and Methods:

Cell Handling

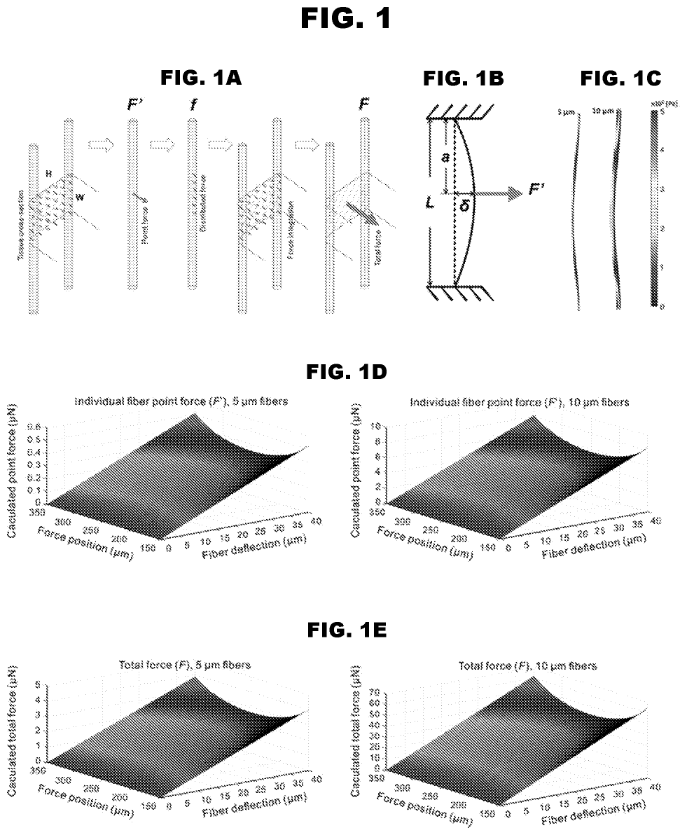

The committee on Human Research at the University of California, San Francisco (UCSF) approved the iPS cell-research protocol. The isogenic cell lines were engineered using TALENs from the wild-type (WT) hiPS cell genetic background. The isogenic heterozygous GCaMP6f knockin (KI) hiPS cell line was generated by inserting GCaMP6f open reading frame into AAVS 1 locus under control of CAG promoter (Tohyama, S. et al., Cell Stem Cell, 2013, 12(1):127-137; Huebsch, N. et al., Sci Rep., 2016, 6:24726). The isogenic homozygous MYBPC3 null hiPS cell line was generated by inserting an artificial early stop codon into exon 1 of MYBPC3, which resulted in early transcript termination (FIG. 1C). Stable clones were selected using Puromycin (0.5 μg/ml). The iPS cells were maintained on 6-well plates coated with growth factor reduced Matrigel in Essential 8 (E8) media (Life Technologies).

FIG. 1 depicts theoretical force calculation based on fiber deflection. (FIG. 1A) Schematic of the total force (F) calculation based on the assumption that force (f) was evenly distributed throughout the tissue cross-section. (FIG. 1B) Schematic of the individual fiber point force (F′) calculation based on fiber deflection. (FIG. 1C) COMSOL simulation showed von Mises stress generated by applying 1 μN and 10 μN forces on individual 5 μm and 10 μm diameter fibers respectively. (FIG. 1D) Theoretical calculation of individual fiber force (F′) with different force positions (a) and fiber deflections (δ) for individual 5 μm and 10 μm fibers. (FIG. 1E) Theoretical calculation of total force (F) with different force positions (a) and fiber deflections (δ) for 5 μm and 10 μm fiber matrices.

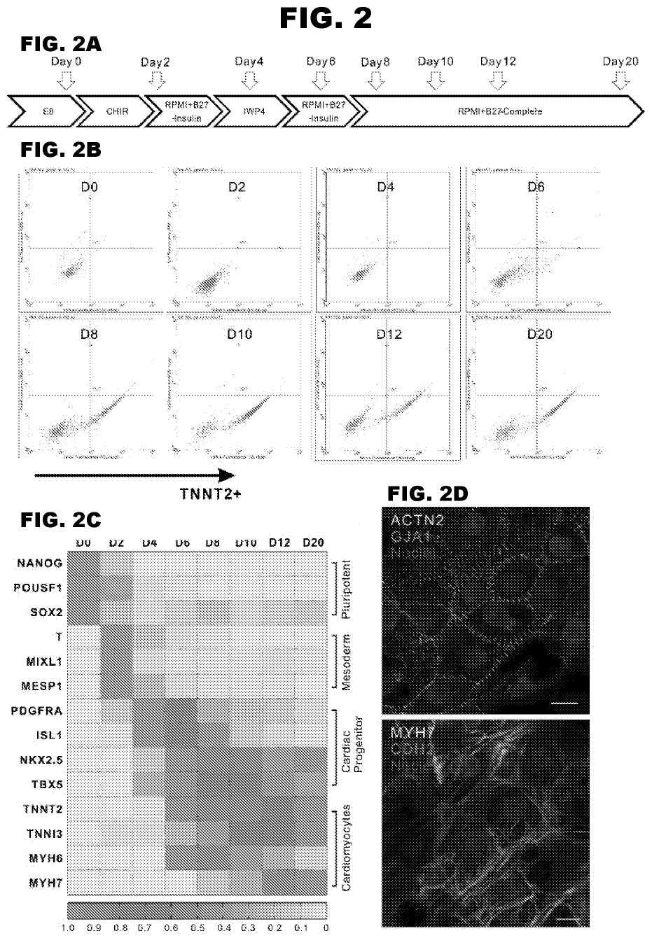

The existing method for monolayer production of hiPS-CMs (Lian, X. et al., Nat Protoc, 2013, 8(1):162-175) was modified (FIG. 2A). WT or genetically modified hiPS cells were plated at a density of 1.25-5×10⁴ cells/cm² onto Matrigel coated 12-well plates in E8 with 10 μM Y-27632 (Stemgent). hiPS cells were maintained in E8 for two additional days, and then started the differentiation “Day 0” with one treatment of 10 μM WNT agonist CHIR99021 (CHIR, Stemgent) in RPMI 1640 media containing B27 supplement without insulin (RPMUB27-I, Life Technologies). After 24 hour CHIR treatment, the cells were maintained in RPMUB27-I media for one day, and then treated with 5 μM WNT inhibitor IWP-4 (Stemgent) in RPMUB27-I media for two days. Subsequently, on Day 5, the media was exchanged to RPMUB27-I for two days and replaced with RPMI 1640 media containing B27 complete supplement (RPMUB27+C) for the continuous culturing.

FIG. 2 depicts the characterization of hiPS-CMs differentiation. (FIG. 2A) The cardiac differentiation was characterized at eight different stages from Day 0 to Day 20. (FIG. 2B) The flow cytometry analysis showed that cardiac differentiation started to produce TNNT2+ cells on Day 6. (FIG. 2C) The gene expression profiling showed cell fate transition from pluripotent stem cells to mesoderm, to cardiac progenitor, and finally to CMs (n=4). (FIG. 2D) hiPS-CMs expressed cardiac specific sarcomere markers (ACTN2 and MYH7) and junctional markers (GJA1 and CDH2). Scale bar, 10 μm.

Flow cytometry analysis of cardiac troponin T (TNNT2) showed the increase of cTnT+ cells starting from Day 6 to the final CM purity ranging from 50% to 70% (FIG. 2B). The gene expression profiling confirmed the cell fate transiting from pluripotency, to mesodermal cells, to cardiac progenitors and finally to CMs (FIG. 2C). The monolayer sheet of hiPS-CMs vigorously beat in the tissue culture plates, and contraction motion could be monitored and analyzed by the motion-tracking software (Huebsch, N. et al., Sci Rep., 2016, 6:24726). The hiPS-CMs expressed cardiac specific sarcomere markers (α-actinin and myosin heavy chain) and junctional markers (connexin43 and N-cadherin) (FIG. 2D).

To enrich hiPS-CMs to ˜80% of total cell population, the existing biochemical purification protocol (Tohyama, S. et al., Cell Stem Cell, 2013, 12(1):127-137) was modified. On Day 15, sheet-beating hiPS-CMs were singularized by a 45-minute treatment of collagenase II (Worthington Biochemical Corp.) in Hanks' balanced salt solution (HBSS, Life Technologies) and a following 2 minutes treatment of 0.25% trypsin, quenched with EB20 media (Knockout DMEM media supplemented with 20% fetal bovine serum (FBS), 1×L-glutamine, 1×MEM non-essential amino acids (MEM-NEAA), 400 nM 2-mercaptoethanol, and 10 μM Y-27632), and replated onto Matrigel-coated 6-well plates in RPMI/B27+C media. After two days recovery in RPMUB27+C media, cells were treated with glucose depleted DMEM media supplemented with 4 mM lactate (Sigma Aldrich) for two days. Purified hiPS-CM were cryopreserved in 90% FBS containing 10% DMSO and 10 μM Y-27632 with cell density of 2 million cells per mL.

Fabrication of Filamentous Matrices

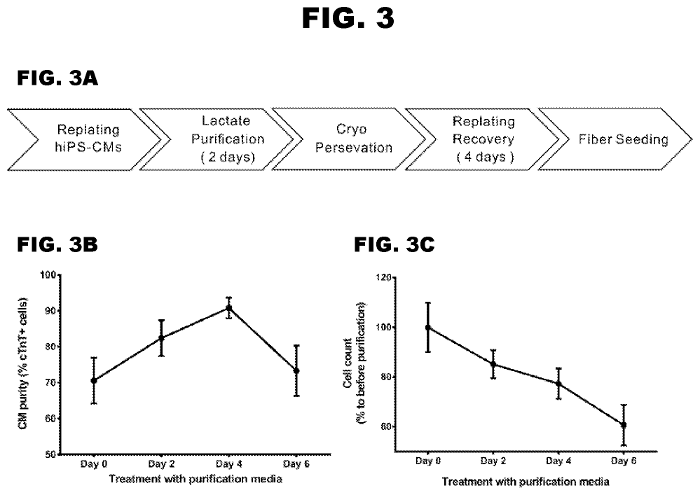

The filamentous matrices were fabricated by two-photon polymerization of photo-curable organic-inorganic hybrid polymer ORMOCLEAR® (Micro resist technology). Briefly, ORMOCLEAR® resin was firstly spin-coated, pre-baked and UV cured on the glass coverslips. Two glass coverslips with cured ORMOCLEAR® thin layers were assembled as one set with 50011m-thick spacer and filled with uncured ORMOCLEAR®. Individual fiber was fabricated by the single radiation to the uncured ORMOCLEAR® through a high repetition rate femtosecond laser (pulse duration:˜400 femtosecond, repetition frequency: 1 MHz, wavelength: 1045 nm, FCPA μJewel D-400, IMRA America, Inc.). The laser beam was frequency-doubled (wavelength: 523 nm) by Lithium triborate (LBO) second harmonic nonlinear crystal (Newlight photonics) and focused at the interface between glass coverslip and ORMOCLEAR® with 5× objective (N.A.=0.14) (M Plan Apo, Mitutoyo). Fiber diameter was determined by the laser power and exposure duration, which was controlled through mechanical shutter. 5 μm fibers were fabricated by 3.7 mW power laser radiation for 0.9 seconds, whereas 10 μm fibers were fabricated by 5.2 mW for 2 seconds. Fiber spacing was controllable by a 3D axis motorized stage with high precision of positioning (Aerotech, ANT95-XY-MP for X-Y axis and ANT95-50-L-Z-RH for Z axis). To fabricate several matrices within one set, the laser radiation was shut during the movement from the end point of the previous matrix to the starting position of next matrix (FIG. 3B). After fiber fabrication, samples were hard-baked for 10 minutes, developed for 30 minutes with a mixture of 2-Isopropyl alcohol and 4-Methyl-2-pentanone (1:1, Sigma Aldrich), rinsed with 2-Isopropyl alcohol, and dipped in 70% ethanol for sterilization.

FIG. 3 depicts the generation of 3D cardiac microtissues on filamentous matrices. (FIG. 3A) The standard hiPS-CMs handling procedure to ensure defined cell population and consistent cell processing for generation of cardiac microtissues. (FIG. 3B) During the purification treatment, the CM purify (cTnT+ cells) increased from Day 0 to Day 4, but decreased at Day 6, while (FIG. 3C) the cell count relative to total cell number before purification decreased over time (mean± SD, n=4).

Generation of 3D Cardiac Microtissues

Three sets of glass devices were placed into one well of a 6-well plate, rinsed with Dulbecco's phosphate buffered saline (DPBS, Gibco) three times, and coated with Matrigel for at least 1 hour. Cryopreserved hiPS-CMs were thawed into EB20 media and plated onto Matrigel-coated 6-well plate with RPMUB27+C media supplemented with 10 μM Y-27632. After 4 days recovery in RPMUB27+C media, the cells were singularized by 0.25% trypsin, quenched with EB20 media, and seeded into filamentous matrices with cell density of 3 million cells per mL RPMUB27+C media supplemented with 10 μM Y-27632. After four hours, another 4 mL RPMUB27+C media supplemented with 10 μM Y-27632 was added into each well to cover the whole set of filamentous matrices. The media was switched to RPMUB27+C media on next day and changed every 2 days. Cardiac tissue beating was recorded every 5 days for motion tracking analysis and force measurement.

Motion Tracking Analysis

Cardiac tissue beating at 100 frames per second was recorded for 10 seconds using a Nikon Eclipse TS 100F microscope with temperature-controlled stage and Hamamatsu ORCA-Flash4.0 V2 digital CMOS camera. Videos of beating cardiac microtissues on both 2D culture dish and 3D filamentous matrices were exported as a series of single-frame image files and analyzed using in-house developed motion-tracking software based on MATLAB (Huebsch, N. et al., Sci Rep., 2016, 6:24726). The software can automatically output the motion heatmap and contraction waveform for calculation of beat rate and maximal contraction velocity. The software is available at “http” followed by “://gladstone.ucsf” followed by “.edu/46749d811/”.

Calcium Flux Recording

For calcium imaging, GCaMP6f hiPS-CMs were differentiated, purified, cryopreserved and seeded into the filamentous matrices for continuous calcium imaging for 20 days. The calcium flux images were recorded at 40 frames per second for 10 seconds using a Nikon Eclipse TS 100F microscope with temperature-controlled stage and Hamamatsu ORCA-Flash4.0 V2 digital CMOS camera.

Fiber Characterization

The elastic modulus (E_{f}) of the fiber was measured by atomic force microscopy (AFM, XE-100, Park Systems) with tip-less AFM cantilevers (TL-CONT-SPL and TL-FM-SPL, Nanosensors). Fiber shape was assumed as a cylinder with a circular cross-section. The Young's modulus of the fiber can be calculated with equation (1).

$${E}_{f} = \frac{64 {Kd}_{c} {L}^{3}}{3 π {D}^{4} ( {d}_{f} - {d}_{c} )} \qquad ( 1 )$$

with length (L) and diameter (D) of the fiber, deflection of the AFM cantilever (dc) and relative deflection of the fiber (df). The spring constants (K) of tip-less AFM cantilevers were measured using the thermal tuning method (Hutter, J. L. and J. Bechhoefer, Review of Scientific Instruments, 1993, 64(7):1868-1873) and calculated to be 0.0636 N/m using AFM software (XEI, Park system). Finally, Young's modulus of the fibers with both 5 μm and 10 μm diameters was calculated as 183.9± 11.7 MPa.

Force Measurement

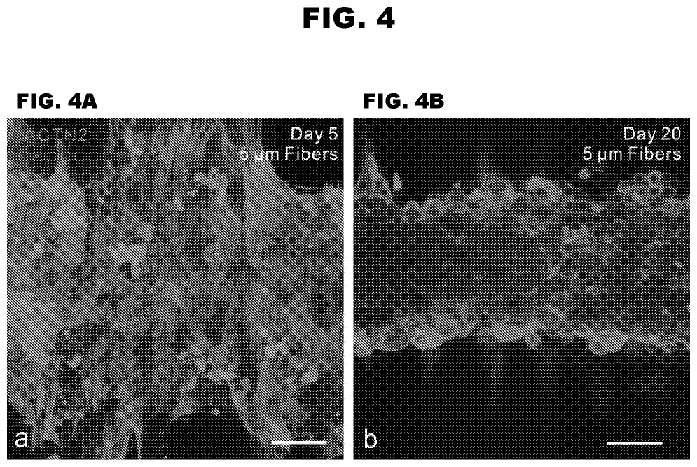

To calculate the contraction forces of the whole cardiac tissue, three assumptions were made: (1) the forces evenly distribute across the tissue cross-section (tissue width multiplying tissue thickness W·H); (2) all the force vectors are parallel each other and perpendicular to the fiber axis; and (3) the fiber has a circular cross-section. Based on those assumptions, the distributed forces (f) were integrated along the fiber as a point force at the position with maximal fiber deflection (FIG. 4A), so that the individual fiber point force (F′) could be calculated based on the beam theory (FIG. 4B). Using the series of single-frame image recorded for cardiac tissue beating, the fiber deflection (8) and its force position (a) can be measured between two consecutive images, so that the point force (F′) applying to the fibers based on the equation (2) can be calculated with Young's modulus (E_{f}), length (L) and diameter (D) of the fiber.

$$F = \frac{3 π {E}_{f} {D}^{4} {( 2 a + L )}^{2}}{{128}_{a}^{3} {( L - a )}^{2}} δ \qquad ( 2 )$$

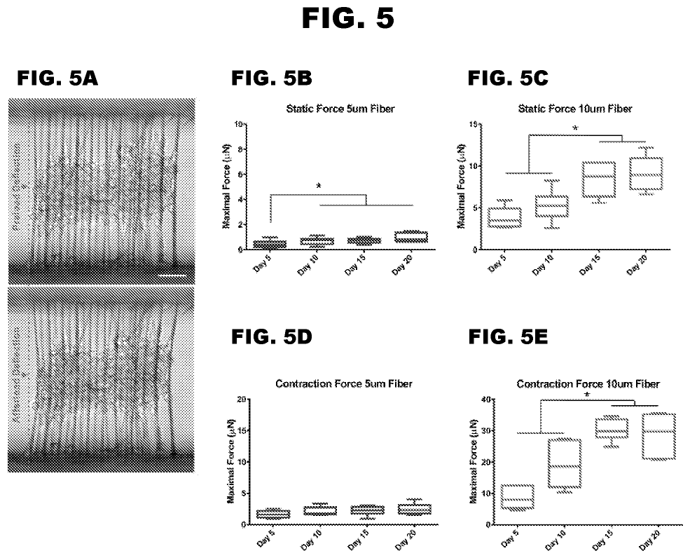

Then, the distributed force (f) can be calculated by dividing the point force (F′) by the area of the force applying to the fiber (tissue width multiplying fiber diameter W·D). Finally, integrating the distributed force across entire tissue cross-section, the total force generated by the cardiac microtissues can be calculated. The static force was calculated based on the preload fiber defection that was measured with the diastolic cardiac tissue in the resting state, whereas the contraction force was calculated based on the afterload fiber defection that was measured with systolic cardiac tissue at maximal contraction (FIG. 5A).

FIG. 4 depicts the cardiac microtissues assembled on filamentous matrices. The confocal fluorescent images showed the cardiac microtissues assembled on a 5 μm fiber matrix at (FIG. 4A) Day 5 and (FIG. 4B) Day 20. Scale bar, 100 μm.

FIG. 5 depicts the mechanical environment altered contractile phenotype. (FIG. 5A) The force development of MYBPC3 deficient cardiac microtissues on 5 μm matrices was faster than WT tissues, but there was no difference in the magnitude. (FIG. 5B) Higher power output of MYBPC3 deficient cardiac microtissues compared to WT suggesting a hyper-contractile phenotype. (FIG. 5C) The force development of MYBPC3 deficient cardiac microtissues on 10 μm matrices was faster and smaller than WT tissues. (FIG. 5D) Lower power output of MYBPC3 deficient microtissues compared to WT suggested an impaired contractile phenotype. For all figures, mean± SD, n=8.

Finite Element Modeling

Finite element modeling for force-induced beam deflection was performed using COMSOL for both 5 μm and 10 μm diameter fibers with length of 500 μm. Fibers were modeled with Young's modulus of 183.9 MPa based on the AFM measurement, and discretized into hexahedral mesh elements. Two ends of fiber were assigned with fixed boundary condition, and the other area was assigned with free boundary condition. The distributed contraction forces (1 μN for 5 μm diameter and 10 μN for 10 μm diameter fibers) were applied perpendicularly to fiber axis with length of 200 μm to the center of the fibers. The maximal stresses were calculated based on maximal deflection of the fiber at the maximal contraction of the cardiac microtissues (FIG. 4C). Transient stress on 10 μm fiber was also calculated based on the temporal deflection of the fiber during the cardiac tissue contraction for 10 seconds.

Immunostaining and Microscopy

Samples were fixed with 4% (vol/vol) paraformaldehyde (PFA) for 15 min, permeabilized with 0.2% Triton-X-100 for 5 min, and blocked with 2% BSA, 4% goat serum and 0.1% Triton-X-100 for 30 min. The samples were then incubated with primary antibodies (Table 1) for 2 hours and secondary antibodies for 1.5 hours. DAPI was used to stain cell nuclei in monolayer cell culture and To-Pro-3 was used for filamentous matrices, because the fiber material was auto-fluorescent under UV excitation. For bright-field and epi-fluorescent microscopy, the images were taken using a Nikon Eclipse TS 100F microscope with Hamamatsu ORCA-Flash4.0 V2 digital CMOS camera. For confocal microscopy, the images were taken with a Zeiss LSM710 laser-scanning microscope the in Biological Imaging Facility (BIF) at UC Berkeley.

TABLE 1
Primary Antibodies

| Antibodies | Dilution | Animal | Vendor | Cat. No. |
|---|---|---|---|---|
| Sarcomeric α-actinin | 1:200 | Mouse | Sigma Aldrich | A7811 |
| Cardiac troponin T | 1:200 | Mouse | Thermo Scientific | MS295P |
| β-myosin heavy chain | 1:200 | Mouse | Abcam | ab97715 |
| Connexin 43 | 1:100 | Rabbit | Sigma Aldrich | C6219 |
| N-cadherin | 1:100 | Rabbit | Abcam | ab12221 |
| Nuclei (DAPI) | 2 drops | — | Life Technologies | R37606 |
| | per mL | | | |
| Nuclei (Topro3) | 1:50 | — | Life Technologies | T3605 |

RT-qPCR

Gene expression profiling was performed using RT-qPCR with customized target arrays for cardiac differentiation (SA Biosciences/Qiagen) and commercial available TaqMan arrays for human NFAT & cardiac hypertrophy (ThermoFisher Scientific) (see, Table 2 below). Cells were washed with DPBS, homogenized and RNA purified using RNeasy Mini Kit for 2D cell culture and RNeasy Micro Kit (Qiagen) for filamentous matrices. To ensure enough amount of RNA for analysis, four sets of matrices under the same conditions were combined as one sample. The total RNA concentration was quantified using a Nanodrop and integrity was determined using the Agilent BioAnalyzer. Conversion of total RNA to cDNA was carried out using SuperScript III Reverse Transcriptase (Life Technologies) with random primers. qPCR was performed on the Applied Biosystems StepOnePlus instrument using 10 ng cDNA per reaction and SYBR Green ROX Master Mix (Qiagen) for customized target arrays and TaqMan fast universal PCR Master Mix for TaqMan arrays (Life Technologies). The data was analyzed using −ΔCt method relative to level of the housekeeping gene. To profile the transient gene expression during the cardiac differentiation, the expression of each gene at different days (Day 0-12) was normalized to the maximal expression of this gene during the differentiation process.

TABLE 2
PCT Transcripts in the Customized Target Array for Cardiac Differentiation Profiling
Cardiac Differentiation Profiling

| NANOG | POUSF1 | SOX2 | T | MIXL1 | MESP1 |
|---|---|---|---|---|---|
| NM_024865 | NM_002701 | NM_003106 | NM_003181 | NM_031944 | NM_018670 |
| PDGFRA | ISL1 | NKX2.5 | TBX5 | TNNT2 | TNNI3 |
| NM_001202 | NM_002202 | NM_004387 | NM_000192 | NM_000364 | NM_000363 |

| MYH6 | MYH7 | GAPDH | HSP90AB1 |
|---|---|---|---|
| NM_002471 | NM_000257 | NM_002046 | NM_007355 |

Flow Cytometry

The efficiency of cardiac differentiation and purification was evaluated using flow cytometry. Cells were singularized with 0.25% trypsin for 5 minutes and quenched with EB20 media. After washing with DPBS three times, cells were fixed with PFA for 15 min, and incubated with primary antibody (mouse monoclonal cardiac Troponin T, Thermo Scientific) and secondary antibody (Alexa488, Life Technologies) for 30 min each in Wash/Permeabilization buffer. The labeled cells were analyzed by Guava easyCyte™ Flow Cytometer (EMD Millipore) in the Stem Cell Shared Facility at UC Berkeley.

Quantitative Sarcomere Analysis

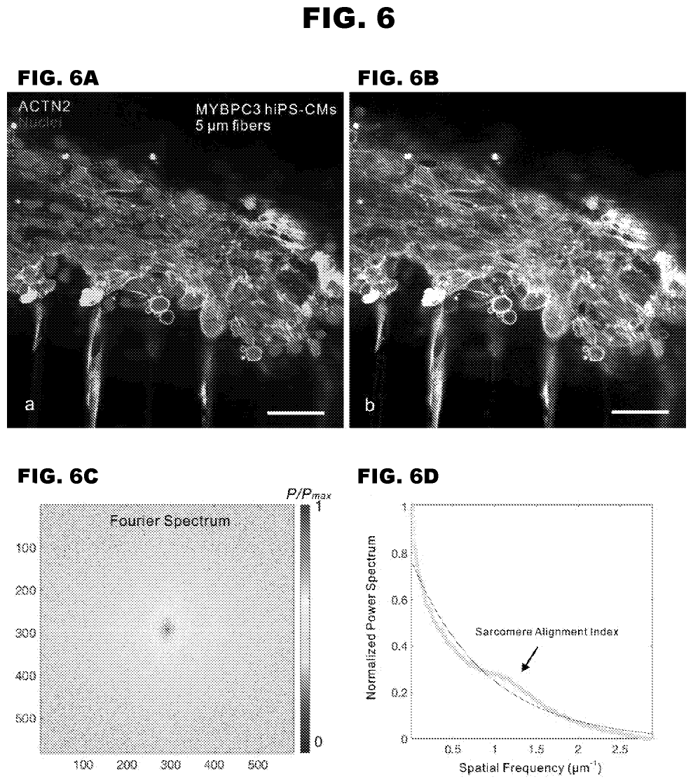

A high-throughout, automated and quantitative analysis on sarcomere alignment was performed using an image-processing algorithm on 2D Fast Flourier transform (2D FFT) (Pasqualini, F. S. et al., Stem Cell Reports, 2015, 4(3):340-347) (FIG. 6A, FIG. 6B). Well-aligned myofibrils in hiPS-CMs contain a spatially repeating pattern of sarcomere with a certain frequency, which can be extracted as a periodic signal by 2D FFT. Most of the energy in the frequency domain is present in the center of the image, which corresponds to the low frequency data in the image. The peak bands away from the center peak corresponding to the high-frequency data represented as the signal from aligned sarcomere (FIG. 6C). These high-frequency peak values can be extracted to compute the “Sarcomere Alignment Index”, which gives a quantitative measurement of the level of sarcomere alignment (FIG. 6D).

FIG. 6 depicts the calculation of sarcomere alignment index. (FIG. 6A) Fluorescent image of a MYBPC3 deficient cardiac microtissue assembled on 5 μm matrices, in which (FIG. 6B) the sarcomere image of ACTN2 was used to compute the sarcomere alignment index. Scale bar, 100 μm. (FIG. 6C) The high-frequency peak bands in Fourier spectrum image represented organized sarcomere in the fluorescent image of ACTN2. (FIG. 6D) This high-frequency peak values can be extracted to compute the “sarcomere alignment index”.

Tension Index Analysis

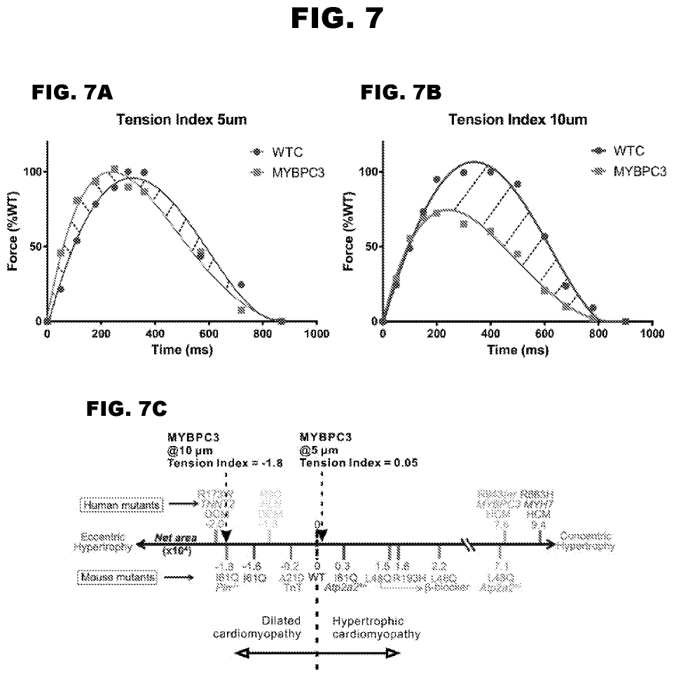

A computational model of integral of myofilament tension has been used to predict HCM and DCM in mice associated with essentially any sarcomeric gene mutation, but also accurately predicts human cardiac disease phenotypes from data generated in hiPS-CMs from familial cardiomyopathy patients. DCM is represented by negative values of the integrated tension index, while positive values represent HCM. To calculate the tension index for our MYBPC3 deficient cardiac microtissues on either 5 μm matrices or 10 μm matrices, the force development kinetics for WT and MYBPC3 deficient cardiac microtissues was first averaged. Second, the averaged force kinetics was normalized to the maximal force of WT cardiac microtissues, and curve-fitted the normalized force kinetics (FIG. 7A, FIG. 7B). Last, the tension index was calculated by subtracting the curve area of WT normalized force kinetics from the curve area of MYBPC3 deficient cardiac microtissues.

FIG. 7 depicts the tension indices for MYBPC3 deficient cardiac microtissues. The normalized force kinetics curves were curve-fitted and the tension indices were calculated for MYBPC3 deficient cardiac microtissues assembled on (FIG. 7A) 5 μm matrices and (FIG. 7B) 10 μm matrices. (FIG. 7C) Calculated tension indices were compared to the other studies on mouse models and patient-derived hiPS-CMs models to distinguish the HCM and DCM phenotypes.

Statistical Analysis

All statistical analysis was performed in GraphPad Prism. Data were presented as mean± SD. For single comparisons, a two-sided Student's t-test was used. For multiple comparisons, one-way analysis of variance was used with post-hoc Tukey tests. p<0.05 was considered significant.

### Example 1: Matrix Fabrication and Cardiac Microtissue Self-Assembly and Remodeling

The filamentous matrices were fabricated using two-photon polymerization (TPP) that produced scaffolds with accurately defined micro and nano-scale features (FIG. 8A) (Kawata, S. et al., Nature, 2001, 412(6848):697-698; Klein, F. et al., Adv Mater, 2010, 22(8):868-871; Jeon, H. et al., J Biomed Mater Res A, 2010, 93(1):56-66). Based on previous studies, the 3-D filamentous fiber matrix, consisting of parallel fibers, with 500 μm fiber length in Y-axis, 50 μm fiber spacing in X-axis, and 30 μm layer spacing in Z-axis robustly generated 3D condensed cardiac microtissues (Ma, Z. et al., Biomaterials, 2014, 35(5):1367-1377) (FIG. 8B). Multiple matrices were fabricated within one pair of glass slides by separating cohorts of fibers with 2 mm matrix spacing in X-axis (FIG. 8B). This design not only increased the throughput, but also made the fiber deflection easier to measure for contraction force calculations. Scanning electron microscopy confirmed a matrix with parallel fibers, and the ability to control fiber diameter (e.g., 5 μm and 10 μm) (FIG. 8C).

FIG. 8 depicts the fabrication of filamentous matrices. (FIG. 8A) Schematics of two-photon polymerization system to fabricate the filamentous matrices. (FIG. 8B) The schematic of one set of filamentous matrices with definitions of fiber spacing, layer spacing and matrix spacing. (FIG. 8C) Bright-field image of top view of a fabricated 3-D filamentous fiber matrix (upper left), SEM images of side view (bottom left) and top view (insertion) of a 3-D filamentous fiber matrix, and SEM images of individual 5 μm and 10 μm fibers. Scale bar, 100 μm (left) and 10 μm (right).

hiPS-CMs were seeded onto filamentous matrices without any external hydrogels. Generation of 3D cardiac microtissues required a relatively purified hiPS-CM population and consistent cell handling procedures (FIG. 3A). It has been reported that a biochemical purification procedure (Burridge, P. W. et al., Nature Methods, 2014, 11(8):855-860; Tohyama, S. et al., Cell Stem Cell, 2013, 12(1):127-137) can result in highly purified CM population (cTnT+ cells >90%). Previous studies on engineered cardiac microtissues suggested the need for stromal cell population to enhance the mechanical integrity and connectivity of tissues (Thavandiran, N. et al., Proc Natl Acad Sci USA, 2013, 110(49):E4698-4707; Huebsch, N. et al., Sci Rep, 2016, 6:24726). Instead of four days of treatment with lactate purification media, cells were treated for two days, which resulted in a mixed hiPS-CMs population (TNN2+ cells ˜80%, FIG. 3b). It was found that continual purification for six days would significantly decrease the cell number (FIG. 3C).

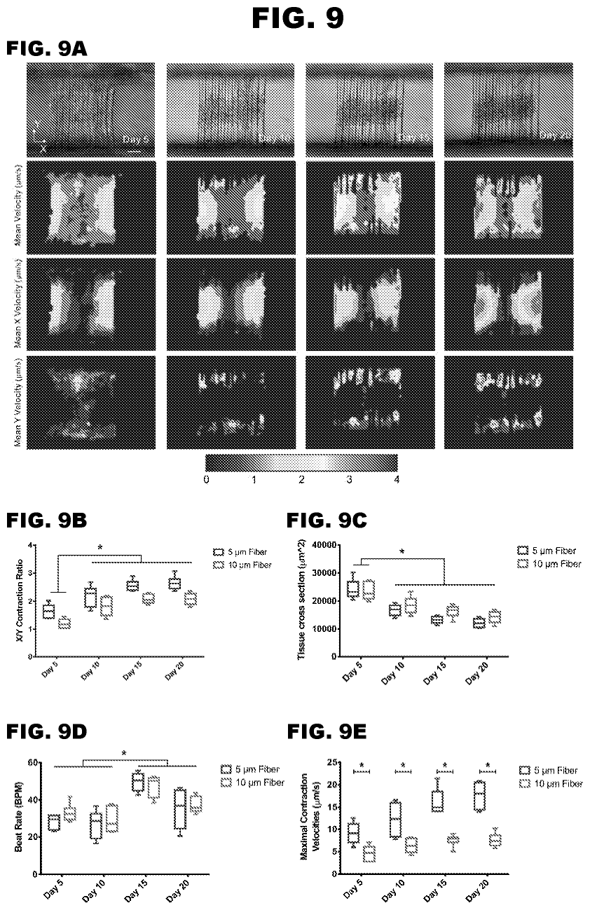

The hiPS-CMs seeded on the filamentous matrices were able to self-assemble into 3D cardiac microtissues (Z-axis thickness˜60 μm, FIG. 4A, FIG. 4B) and maintained a stable beat rate after 5-days of culturing. The cardiac microtissues continuously and progressively remodeled in response to the passive mechanical resistance of the fibers and active tissue contraction. The beat rate, contraction velocity, contraction force, and tissue width of the individual cardiac microtissues were measured every 5 days to track the tissue remodeling associated with the change of functional readouts. Since the individual fibers were fixed onto the glass slides at both ends, the contracting cardiac microtissues were able to deform the fibers in the x-direction, but not in y-direction. This mechanical constraint resulted in the anisotropic contraction with higher contraction along the X-axis than the Y-axis, as defined by contraction heatmaps (FIG. 9A). The ratio of contraction velocity in the X and Y directions was calculated and a significant increase in the ratio from Day 5 to Day 20 was found (FIG. 9B). It was also observed that the microtissues condensed in the direction of the Y-axis, but maintained the integrity along the X-axis, resulting in a significant decrease in the tissue cross-section areas (Z-Y direction) from Day 5 to Day 10-20 (FIG. 9C).

FIG. 9 depicts cardiac microtissues remodeling on filamentous matrices. (FIG. 9A) WT Cardiac microtissues on a 5 μm fiber matrix remodeled tissue shape from Day 5 to Day 20, and the contraction heatmaps showed anisotropic contraction with higher contraction in the X-direction compared to the Y-direction. Scale bar, 100 μm. The progressive tissue remodeling manifested as (FIG. 9B) an increase of the ratio of mean contraction between X-axis and Y-axis and (FIG. 9C) a decrease of the tissue cross-section (Z-Y direction) by comparing Day 5 to Day 10-20 (mean± SD, n=8). By investigating the effect of tissue mechanical environment on cardiac contractility, (FIG. 9D) no significant difference was found on beat rate, but (FIG. 9E) much higher maximal contraction for the cardiac microtissues assembled on 5 μm matrices than the ones on 10 μm matrices (mean± SD, n=8).

### Example 2: Tissue Mechanical Environment Affected Cardiac Tissue Function

To demonstrate the effect of microenvironment mechanics on cardiac tissue function, matrices were created with resistance to contraction. By changing the fiber diameter, the fiber bending stiffness could be changed to modulate the mechanical resistance to the cardiac microtissues. Based on AFM calibration of individual fibers, the elastic modulus of the material was calculated as 183.9± 11.7 MPa, which refers to the linear ratio of force load and deformation of the fiber. Although the elastic modulus is the same for both fibers, the fiber bending stiffness, the mechanical resistance to the cardiac microtissue contraction, is proportional to the square of fiber diameter, thus 5 μm fibers are much easier to bend compared to 10 μm fibers. No significant difference was observed in the beat rate of the cardiac microtissues on filamentous matrices with fiber diameters of 5 μm and 10 μm (FIG. 9D), but the beat rate slightly increased from Day 5-10 to Day 15-20. Since 5 μm fibers were easier to be bend, higher maximal contraction velocity for cardiac microtissues assembled on the 5 μm matrices was found compared to 10 μm matrices (FIG. 9E).

The deflection of individual fibers was used to calculate the force of contraction. By assuming all the forces throughout the tissue cross-section were evenly distributed and parallel (FIG. 1A), the point force exerted on individual fiber was calculated based on the fiber deflection and force position (where the deflection locates) measured in a series of recorded images (FIG. 1B). Using this point force, the total force generated by the cardiac microtissues could be calculated. Through theoretical calculations, the force measured by 10 μm fiber was found to be around 10-fold higher than the force measured by 5 μm fiber with the same fiber deflection and force position (FIG. 1D, FIG. 1E). Therefore, artificially applying the forces at the center region of the fiber with 1 μN to a 5 μm fiber and 10 μN to a 10 μm fiber, the stress generated on the fibers could be determined through COMSOL numerical simulation. High stress occurred at the center region of the fiber, where the force was applied, and also occurred at the two ends of the fiber, where the fiber was fixed at the glass slides (FIG. 1C).

Cardiac preload is defined as end-diastolic myocardial wall tension. Preload is referred to as the passive tension exerted by the fibers at two edges of the matrix to the diastolic cardiac microtissue in the resting state. The load opposing shortening of the ventricular muscles is termed cardiac afterload. The cardiac tissue afterload is defined as the fiber tension induced by the systolic cardiac microtissue at the maximal contraction (FIG. 5A). The afterload is considerably increased when the cardiac microtissues have to beat against stiffer fibers. Based on the fiber deflections and two-end fixed beam theory, the static forces (diastole) and contraction forces (systole) were calculated for the cardiac microtissues assembled on both 5 μm and 10 μm diameter filamentous matrices, and it was found that cardiac microtissues produced higher forces when grown on the matrices with high resistance fibers. It was found that the static forces increased significantly from Day 5 to Day 20 for the cardiac microtissues assembled on both 5 μm and 10 μm filamentous matrices (FIG. 5B, FIG. 5C), whereas the contraction forces increased significantly when the tissues grew on the 10 μm matrices, not on the 5 μm matrices (FIG. 5D, FIG. 5E). Self-assembled WT hiPS-CMs on the 10 μm filamentous matrices were able to adapt to the high stiffness and increase the contraction force through mechanical conditioning, or exercising.

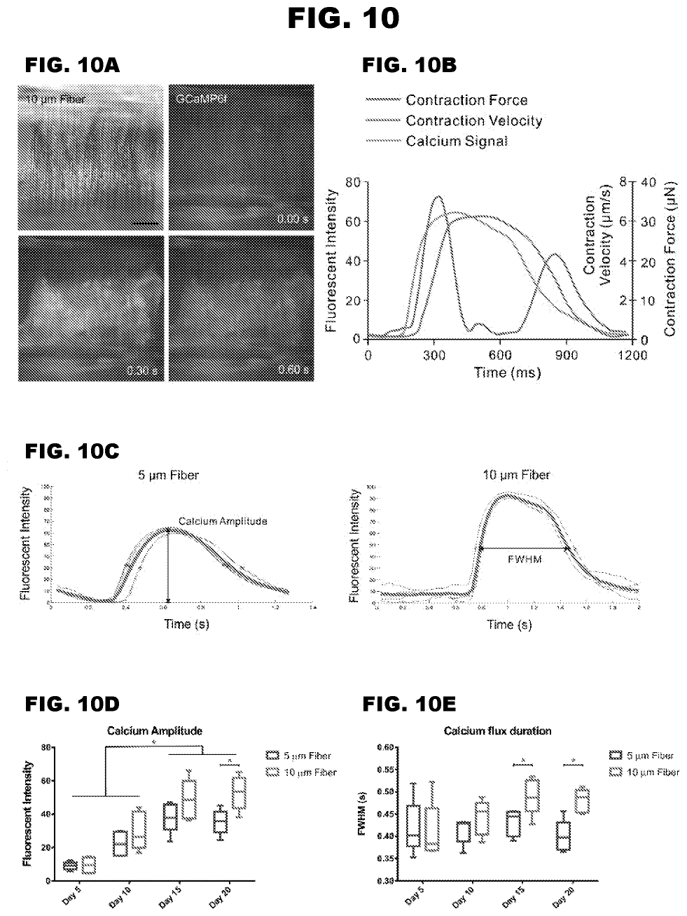

Spontaneous calcium flux in the cardiac microtissues formed by isogenic hiPS-CMs harboring the genetically-encoded Ca²⁺ reporter, GCaMP6f, which was inserted into the AAVS1 locus (Huebsch, N. et al., Tissue Eng Part C Methods, 2015, 21(5):467 479) was monitored. High-speed imaging captured the fluorescent fluctuation of calcium flux from the GCaMP6f cardiac tissue assembled on the filamentous matrices (FIG. 10A). By tracking the contraction motion, fiber deflection, and GCaMP fluorescent signal from the same cardiac tissue, the temporal relationship among contraction velocity, force, and calcium flux was characterized (FIG. 10B). According to the waveform of the calcium flux, the calcium amplitude and full width half maximum (FWHM) was measured as the key electrophysiological properties of the cardiac microtissues assembled on both 5 μm and 10 μm filamentous matrices (FIG. 10C). It was found that the calcium amplitude significantly increased from Day 5 to Day 20 (FIG. 10D). The enhancement of calcium flux duration from the cardiac microtissues correlated with the increase of contraction force on 10 μm matrices, but not on 5 μm matrices. At the late stages Day 15-20, it was observed that cardiac microtissues on 10 μm matrices showed higher calcium amplitude (FIG. 10D) and longer calcium flux duration (FIG. 10E) compared to the ones assembled on 5 μm matrices.

FIG. 10 depicts the calcium flux of the cardiac microtissues. (FIG. 10A) Fluorescent fluctuation of calcium flux of GCaMP6f expressing cardiac microtissues on 10 μm fiber matrices. Scale bar, 100 μm. (FIG. 10B) The contraction velocity, contraction force, and calcium flux fluorescent signal plotted temporally. (FIG. 10C) Representative calcium flux waveforms, indicating the measured calcium amplitude and FWHM for the cardiac microtissues assembled on 5 μm and 10 μm matrices. The cardiac microtissues on 10 μm matrices exhibited (FIG. 10D) higher calcium amplitude at Day 20 and (FIG. 10E) longer calcium flux duration at Day 15 & 20 compared to 5 μm matrices (mean± SD, n=8).

### Example 3: Tissue Mechanical Environment Affected Genetically-Related Contractile Deficits

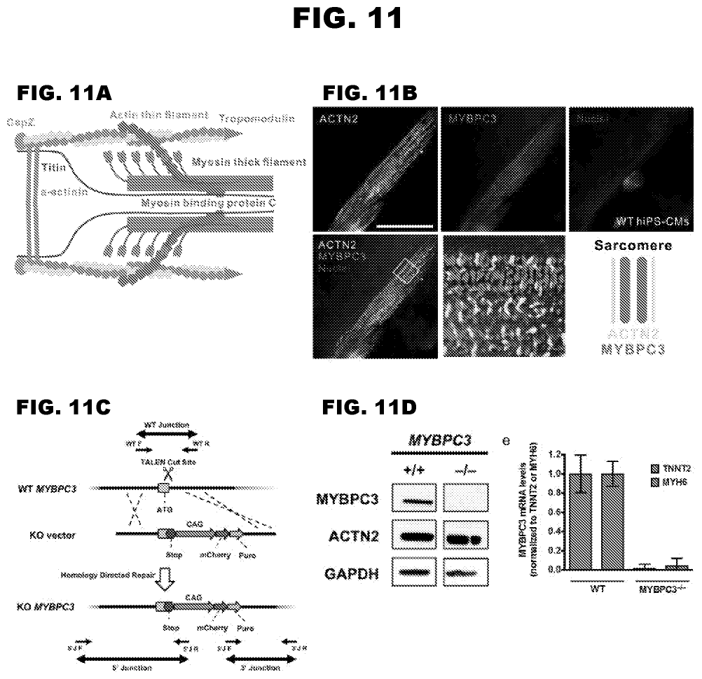

To elucidate how mechanical load can affect the contraction deficits and pathological phenotypes, TALEN-assisted gene-editing was used to knockout MYBPC3 to create a human diseased cardiac tissue model. MYBPC3 is a thick filament associated protein, which is thought to play a principally structural role stabilization of the sarcomere sliding during contraction (Gautel, M. et al., Circ Res, 1998, 82(1):124-129; Bennett, P. M. et al., Rev Physiol Biochem Pharmacol, 1999, 138:203-234) (FIG. 11A). Fluorescent images of WT hiPS-CMs showed the MYBPC3 protein aligned with ATCN2 protein, indicating the structural relationship of A bands and Z discs (FIG. 11B). Furthermore, this protein binds to myosin and actin, thereby regulating the probability of cross-bridge interactions, which in turn controls the rate of force development and relaxation in the cardiac muscles (Moss, R. L. et al., Circ Res, 2015, 116(1):183-192). Mutations in the MYBPC3 gene have been found to increase the risk of heart failure through either HCM or DCM (Flashman, E. et al., Circ Res, 2004, 94(10):1279-1289; Sequeira, V. et al., Pflugers Arch, 2014, 466(2):201-206). The vast majority of patients with heterozygous MYBPC3 gene mutations developed adult-onset HCM, resulting in genetic predisposition for heart failure with risk increased by hypertension, age, and other environmental factors. Homozygous MYBPC3 mutations are rarer in human, but cause severe DCM phenotypes and childhood early death (Jiang, J. et al., Proc Natl Acad Sci USA, 2015, 112(29):9046-9051; Dhandapany, P. S. et al., Nat Genet, 2009, 41(2):187-191).

FIG. 11 depicts the generation of the MYBPC3 null hiPS cell line. (FIG. 11A) Schematic of MYBPC3 protein in one unit of myofibril interacting with thin filaments, thick filaments and titin. (FIG. 11B) Fluorescent images showing structural location of ACTN2 and MYBPC3 proteins of WT hiPS-CMs. Scale bar, 50 μm (FIG. 11C) Schematic of the generation of MYBPC3 null hiPS cell line from WT through TALEN-mediated genome editing. (FIG. 11D) The CMs derived from MYBPC3 null hiPS cells showed (d) absence of MYBPC3 protein production by western blotting and (FIG. 11E) significant reduction of MYBPC3 mRNA expression relative to TNNT2 and MYH6.

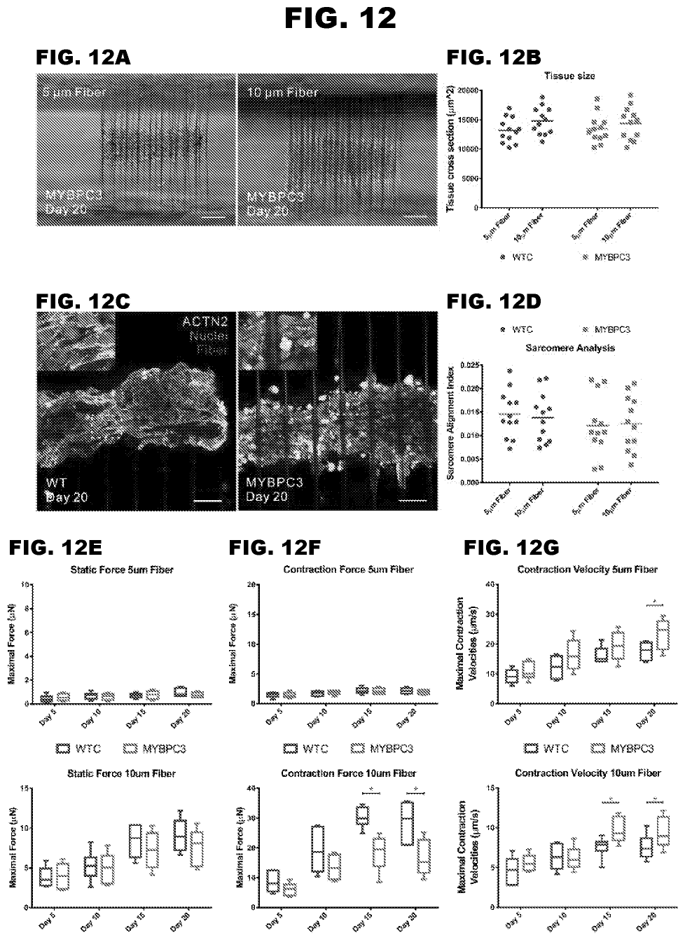

The isogenic homozygous MYBPC3 null hiPS cell line was developed by TALEN-mediated gene-editing methods (FIG. 11C). hiPS-CMs derived from MYBPC3 null hiPS cells showed reduction of MYBPC3 mRNA and protein (FIG. 11D, FIG. 11E). MYBPC3 hiPS-CMs formed the 3D anisotropic cardiac microtissues on both 5 μm and 10 μm filamentous matrices (FIG. 12A). The structural characteristics between WT and MYBPC3 deficient cardiac microtissues on 5 μm and 10 μm diameter matrices was compared (FIG. 12A, FIG. 12C). At Day 20, no significant differences was found on both tissue cross-section areas (FIG. 12B) and sarcomere alignment indices (FIG. 12D) from either of the two tissue or matrix types.

FIG. 12 depicts contraction deficits of MYBPC3 deficient cardiac microtissues. (FIG. 12A) Bright-field microscopy showed (FIG. 12B) no significant difference on tissue cross-section between WT and MYBPC3 deficient cardiac microtissues assembled on 5 μm and 10 μm matrices at Day 20 (mean with all data, n=12). Scale bar, 100 μm. (FIG. 12C) Confocal microscopy showed (FIG. 12D) no significant difference on sarcomere alignment index between WT and MYBPC3 deficient microtissues assembled on 5 μm and 10 μm matrices at Day 20 (mean with all data, n=12). Scale bar, 50 μm. (FIG. 12E) Comparing to WT, MYBPC3 deficient cardiac microtissues showed no significant difference on static forces for the microtissues on both 5 μm and 10 μm matrices, and (FIG. 12F) no difference on contraction forces for the microtissues on 5 μm matrices, but lower contraction forces at Day 15 & 20 for the microtissues only on 10 μm matrices. (FIG. 12G) MYBPC3 deficient cardiac microtissues showed higher maximal contraction velocity for the cardiac microtissues assembled on both 5 μm (Day 20) and 10 μm matrices (Day 15 & 20) compared to WT cardiac microtissues (mean± SD, n=8).

Conditioning and the microenvironment mechanics affect contraction forces in MYBPC3 deficient cardiac microtissues. No significant difference on static forces was found between WT and MYBPC3 deficient cardiac microtissues on both 5 μm and 10 μm matrices (FIG. 12E). The MYBPC3 deficient cardiac microtissues exhibited significantly lower contraction forces compared to WT microtissues only on 10 μm diameter matrices, but not on 5 μm diameter matrices (FIG. 12F). It was found that the MYBPC3 deficient cardiac microtissues showed higher contraction velocity compared to WT, and this velocity difference between WT and MYBPC3 deficient cardiac microtissues was exaggerated on 10 μm matrices (FIG. 12G).

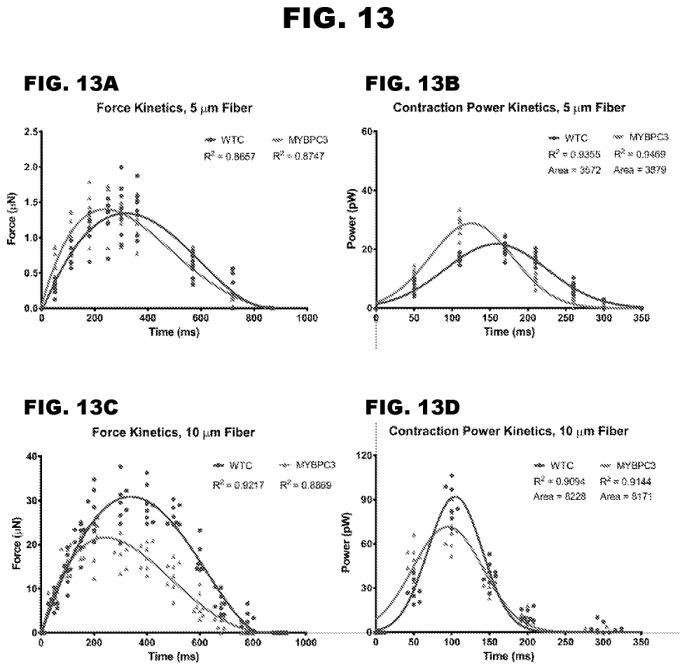

Since MYBPC3 protein was thought to regulate the force development during cardiac contraction, the force kinetics curves was plotted for WT and MYBPC3 deficient cardiac microtissues on 5 μm and 10 μm matrices at Day 20. By multiplying the force and velocity, the power kinetics curves was plotted and the curve area was measured as the total energy consumed by the cardiac microtissues to complete one contraction. On both 5 μm and 10 μm matrices, MYBPC3 deficient cardiac microtissues developed the maximal contraction forces faster than WT microtissues (FIG. 13A, FIG. 13C). Since the force magnitude was similar between WT and MYBPC3 deficient cardiac microtissues on 5 μm matrices, higher contraction velocity led to higher power output and more energy consumption of the MYBPC3 deficient cardiac microtissues (FIG. 13B). This hyper-contractile characteristic has been widely accepted as an early sign of HCM phenotype. On the other hand, MYPBC3 cardiac microtissues developed significantly lower contraction forces, less power output, but similar energy consumption compared to the WT on 10 μm matrices (FIG. 13C, FIG. 13D). The same energy consumption, but low force production from MYBPC3 deficient cardiac microtissues indicated that absence of MYBPC3 protein impaired the contraction of the cardiac microtissues in an energy-efficient manner. This impaired cardiac function possibly recapitulates the failing myocardium with a DCM phenotype due to the genetic deficiency and manifested by external stress.

FIG. 13 depicts the mechanical environment altered contractile phenotype. (FIG. 13A) The force development of MYBPC3 deficient cardiac microtissues on 5 μm matrices was faster than WT tissues, but there was no difference in the magnitude. (FIG. 13B) Higher power output of MYBPC3 deficient cardiac microtissues compared to WT suggesting a hyper-contractile phenotype. (FIG. 13C) The force development of MYBPC3 deficient cardiac microtissues on 10 μm matrices was faster and smaller than WT tissues. (FIG. 13D) Lower power output of MYBPC3 deficient microtissues compared to WT suggested an impaired contractile phenotype. For all figures, mean± SD, n=8.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A three-dimensional filamentous fiber matrix comprising:
a) a first cardiomyocyte population comprising a mutation in a gene encoding a gene product required for normal cardiomyocyte function, wherein the mutation reduces the level or the activity of the gene product; and/or
b) a second cardiomyocyte population, wherein the second cardiomyocyte population is isogenic with the first cardiomyocyte population, but does not comprise the mutation.

2. The matrix of claim 1, wherein the gene product is selected from a cardiac myosin binding protein C polypeptide, a cytoskeletal polypeptide, δ-sarcoglycan (SGCD), β-sarcoglycan (SGCB), desmin (DES), lamin A/C (LMNA), vinculin (VCL), a sarcomeric/myofibrillar polypeptide, α-cardiac actin (ACTC), troponin T (TNNT2), troponin I (TNNI3), β-myosin heavy chain (MYH7), myosin binding protein C (MBPC3), α-tropomyosin (TPM1), a Z-disk protein, muscle LIM protein (MLP), cysteine and glycine-rich protein 3 (CSRP3), titin (TTN), telethonin/TCAP, α-actinin-2 (ACTN2), nebulette (NEBL), myopalladin (MYPN), ANKRD1/CARP, ZASP/LIM-domain binding 3 (LBD3), cardiac sodium channel gene SCN5A, calcium homeostasis regulator phospholamban (PLN), desmoplakin (DSP), desmoglein-2 (DSG2), and desmocolin-2 (DSC2).

3. The matrix of claim 1, wherein the mutation is a loss-of-function mutation.

4. The matrix of claim 1, wherein the first and the second cardiomyocyte populations are human cardiomyocytes.

5. The matrix of claim 1, wherein the first cardiomyocyte population is genetically modified to produce a polypeptide calcium reporter.

6. The matrix of claim 5, wherein the calcium reporter is GCaMP6f.

7. The matrix of any one of claims 1-6, wherein the matrix comprises filamentous fibers having a diameter of from 2 μm to 20 μm.

8. The matrix of any one of claims 1-6, wherein the matrix comprises filamentous fibers having a diameter of from 5 μm to 10 μm.

9. The matrix of any one of claims 1-8, wherein the matrix comprises filamentous fibers, each fiber comprising a first end and a second end, wherein the first end and the second end of the fiber are attached to a solid support.

10. The matrix of claim 9, wherein the solid support comprises glass or a non-water-soluble polymer.

11. The matrix of any one of claims 1-10, wherein the filamentous fibers are from 450 μm to 600 μm in length in the Y-axis.

12. The matrix of any one of claims 1-11, wherein the filamentous fibers form layers spaced from about 40 μm to about 60 μm apart in the X-axis, and wherein the layers are spaced from about 25 μm to about 35 μm in the Z-axis.

13. The matrix of any one of claims 1-12, wherein the filamentous fibers have an elastic modulus of from about 160 MPa to about 200 MPa.

14. The matrix of any one of claims 1-12, wherein the filamentous fibers have an elastic modulus of from about 170 MPa to about 190 MPa.

15. The matrix of any one of claims 1-14, wherein the cardiomyocytes are present in the matrix at a density of from 1×10⁶ cells/cc to 6×10⁶ cells/cc.

16. The matrix of any one of claims 1-14, wherein the cardiomyocytes are present in the matrix at a density of from 2×10⁶ cells/cc to 5×10⁶ cells/cc.

17. A system comprising:
a) a first three-dimensional filamentous fiber matrix comprising a first cardiomyocyte population comprising a mutation in a gene encoding a gene product required for normal cardiomyocyte function, wherein the mutation reduces the level or the activity of the gene product; and
b) a second three-dimensional filamentous fiber matrix comprising a second cardiomyocyte population, wherein the second cardiomyocyte population is isogenic with the first cardiomyocyte population, but does not comprise the mutation,
wherein the first and the second matrices are present on a solid support and separated from one another by a distance of from 1 mm to 5 mm.

18. The system of claim 17, wherein the gene product is a cardiac myosin binding protein C polypeptide.

19. The system of claim 17, wherein the mutation is a loss-of-function mutation.

20. The system of claim 17, wherein the first and the second cardiomyocyte populations are human cardiomyocytes.

21. The system of claim 17, wherein the first cardiomyocyte population is genetically modified to produce a polypeptide calcium reporter.

22. The system of claim 21, wherein the calcium reporter is GCaMP6f.

23. The system of any one of claims 17-22, wherein the first and the second matrix comprises filamentous fibers having a diameter of from 2 μm to 20 μm.

24. The system of any one of claims 17-22, wherein the first and the second matrix comprises filamentous fibers having a diameter of from 5 μm to 10 μm.

25. The system of any one of claims 17-24, wherein the first and the second matrix comprises filamentous fibers, each fiber comprising a first end and a second end, wherein the first end and the second end of the fiber are attached to the solid support.

26. The system of claim 25, wherein the solid support comprises glass or a non-water-soluble polymer.

27. The system of any one of claims 17-26, wherein the filamentous fibers are from 450 μm to 600 μm in length in the Y-axis.

28. The system of any one of claims 17-27, wherein the filamentous fibers form layers spaced from about 40 μm to about 60 μm apart in the X-axis, and wherein the layers are spaced from about 25 μm to about 35 μm in the Z-axis.

29. The system of any one of claims 17-28, wherein the filamentous fibers have an elastic modulus of from about 160 MPa to about 200 MPa.

30. The system of any one of claims 17-28, wherein the filamentous fibers have an elastic modulus of from about 170 MPa to about 190 MPa.

31. The system of any one of claims 17-30, wherein the cardiomyocytes are present in the first and the second matrix at a density of from 1×10⁶ cells/cc to 6×10⁶ cells/cc.

32. The system of any one of claims 17-30, wherein the cardiomyocytes are present in the first and the second matrix at a density of from 2×10⁶ cells/cc to 5×10⁶ cells/cc.

33. The system of any one of claims 17-32, comprising a device for tracking motion of the cardiomyocytes.

34. The system of any one of claims 17-33, comprising a device for measuring deflection of the filamentous fibers in the matrices in response to cardiomyocyte contraction.

35. The system of any one of claims 17-34, comprising a device for measuring force applied by the cardiomyocytes on the filamentous fibers.

36. A method of characterizing a mutation in a gene encoding a gene product required for normal cardiomyocyte function, the method comprising measuring deflection of the filamentous fibers in the matrices in response to cardiomyocyte contraction in a matrix of any one of claims 1-16, wherein the cardiomyocytes comprising a mutation in a gene encoding a gene product required for normal cardiomyocyte function, wherein the mutation reduces the level or the activity of the gene product.

37. A method of identifying a candidate agent for treating a cardiomyopathy, the method comprising:
a) contacting cardiomyocytes in a matrix of any one of claims 1-16 with a test agent, wherein the cardiomyocytes comprising a mutation in a gene encoding a gene product required for normal cardiomyocyte function, wherein the mutation reduces the level or the activity of the gene product; and
b) measuring the effect of the test agent on deflection of the filamentous fibers in the matrix in response to cardiomyocyte contraction, wherein a test agent that increases the deflection, compared to a control, is a candidate agent for treating a myopathy.

38. The method of claim 37, wherein the cardiomyocytes are obtained from an individual with a cardiomyopathy.

39. The method of claim 37, wherein the cardiomyocytes are generated from induced pluripotent stem cells generated from cells obtained from an individual with a cardiomyopathy.


SEQUENCE LISTING (1 sequence)

SEQ ID NO: 1 - protein, 448 aa, Artificial sequence
  synthetic polypeptide
  misc_feature (222)..(222): Xaa can be any naturally occurring amino acid
MGSHHHHHHGMASMTGGQQMGRDLYDDDDKDLATMVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLXVQCFSRYPDHMKQHDFFKSAMPEGYIQERTIFFKDDGNYKTRAEVKFEGDTLVNRIELKGIDFKEDGNILGHKLEYNLPDQLTEEQIAEFKEAFSLFDKDGDGTITTKELGTVMRSLGQNPTEAELQDMINEVDADGDGTIDFPEFLTMMARKGSYRDTEEEIREAFGVFDKDGNGYISAAELRHVMTNLGEKLTDEEVDEMIREADIDGDGQVNYEEFVQMMTAK